SEQUENCE LISTING (39 sequences)

SEQ ID NO: 1 - DNA, 22 bp, Artificial Sequence
  Synthetic - Primer for PR8 probe-based qPCR
gagtgacatcaaaatcatggcg

SEQ ID NO: 2 - DNA, 21 bp, Artificial Sequence
  Synthetic - Primer for PR8 probe-based qPCR
cgttctccatcagtctccatc

SEQ ID NO: 3 - DNA, 24 bp, Artificial Sequence
  Synthetic - Probe for PR8 probe-based qPCR
aggcaccaaacggtcttacgaaca

SEQ ID NO: 4 - DNA, 21 bp, Artificial Sequence
  Synthetic - Primer for PR8 probe-based qPCR
gttcctgttaccctgataccg

SEQ ID NO: 5 - DNA, 18 bp, Artificial Sequence
  Synthetic - Primer for PR8 probe-based qPCR
tcaccgaaaaccccactg

SEQ ID NO: 6 - DNA, 22 bp, Artificial Sequence
  Synthetic - Probe for PR8 probe-based qPCR
tgatcgaagacacccatggccg

SEQ ID NO: 7 - DNA, 19 bp, Artificial Sequence
  Synthetic - Primer for PR8 dye-based qPCR
tgcatttaccgtcgcttta

SEQ ID NO: 8 - DNA, 20 bp, Artificial Sequence
  Synthetic - Primer for PR8 dye-based qPCR
cctcatagactttggcactc

SEQ ID NO: 9 - DNA, 20 bp, Artificial Sequence
  Synthetic - Primer for PR8 dye-based qPCR
gcactacagctaaggctatg

SEQ ID NO: 10 - DNA, 20 bp, Artificial Sequence
  Synthetic - Primer for PR8 dye-based qPCR
gagtcccaatggttctcatc

SEQ ID NO: 11 - DNA, 21 bp, Artificial Sequence
  Synthetic - Primer for PR8 dye-based qPCR
catactgctgaggatgtcaaa

SEQ ID NO: 12 - DNA, 22 bp, Artificial Sequence
  Synthetic - Primer for PR8 dye-based qPCR
cagagactcgaactgtgttatc

SEQ ID NO: 13 - DNA, 20 bp, Artificial Sequence
  Synthetic - Primer for PR8 dye-based qPCR
cttcgccgagatcagaaatc

SEQ ID NO: 14 - DNA, 20 bp, Artificial Sequence
  Synthetic - Primer for PR8 dye-based qPCR
tcagaatccgctccactatc

SEQ ID NO: 15 - DNA, 20 bp, Artificial Sequence
  Synthetic - Primer for PR8 dye-based qPCR
ggagcagtggctgtattaaa

SEQ ID NO: 16 - DNA, 20 bp, Artificial Sequence
  Synthetic - Primer for PR8 dye-based qPCR
cgggccatcagtcattatag

SEQ ID NO: 17 - DNA, 18 bp, Artificial Sequence
  Synthetic - Primer for PR8 dye-based qPCR
gccagaatgccactgaaa

SEQ ID NO: 18 - DNA, 21 bp, Artificial Sequence
  Synthetic - Primer for PR8 dye-based qPCR
gatcaaccgtccctcataatc

SEQ ID NO: 19 - DNA, 21 bp, Artificial Sequence
  Synthetic - Primer for PR8 dye-based qPCR
tcccttttttttcctcaggtg

SEQ ID NO: 20 - DNA, 20 bp, Artificial Sequence
  Synthetic - Primer for PR8 dye-based qPCR
attaaggccctataagggct

SEQ ID NO: 21 - DNA, 21 bp, Artificial Sequence
  Synthetic - Primer for PR8 dye-based qPCR
tcccttttttttcctcaggtg

SEQ ID NO: 22 - DNA, 20 bp, Artificial Sequence
  Synthetic - Primer for PR8 dye-based qPCR
agtacctgcttctcagttca

SEQ ID NO: 23 - RNA, 21 nt, Artificial Sequence
  Synthetic - Exemplary 3′ end of artificial intron in mRNA
uuuucccuuuuuuuuccucag

SEQ ID NO: 24 - DNA, 21 bp, Artificial Sequence
  Synthetic - Exemplary 3′ end of artificial intron in plasmid
ttttcccttttttttcctcag

SEQ ID NO: 25 - RNA, 21 nt, Artificial Sequence
  Synthetic - Exemplary 3′ end of artificial intron in viral segment
cugaggaaaaaaaagggaaaa

SEQ ID NO: 26 - RNA, 125 nt, Artificial Sequence
  Synthetic - constitutively spliced artificial intron (mRNA)
gugaguauaccggucucuaaaagcgggcaugacuucuagaguaguccaggguuuccgaggguuuccgucgacucagcucgucucgagggcguacuaacuaccgguuuucccuuuuuuuuccucag

SEQ ID NO: 27 - DNA, 125 bp, Artificial Sequence
  Synthetic - constitutively spliced artificial intron (plasmid)
gtgagtataccggtctctaaaagcgggcatgacttctagagtagtccagggtttccgagggtttccgtcgactcagctcgtctcgagggcgtactaactaccggttttcccttttttttcctcag

SEQ ID NO: 28 - RNA, 125 nt, Artificial Sequence
  Synthetic - constitutively spliced artificial intron (viral segment)
cugaggaaaaaaaagggaaaaccgguaguuaguacgcccucgagacgagcugagucgacggaaacccucggaaacccuggacuacucuagaagucaugcccgcuuuuagagaccgguauacucac

SEQ ID NO: 29 - RNA, 556 nt, Artificial Sequence
  Synthetic - NanoLuc-encoding artificial intron (mRNA)
gugaguauaccgguaugguauucacucuugaagacuucgugggugauuggcgucaaaccgcagguuacaacuuagaccagguuuuggagcagggugguguaucuucuuuauuucaaaaccuugguguuucaguaacgccaauucaacgcaucguuuuaucaggcgaaaacggccuuaaaaucgauauucacguuaucaucccuuacgagggcuugucaggugaucaaaugggccaaaucgaaaaaaucuuuaaaguaguauauccaguugaugaucaccacuuuaaaguuaucuuacauuacgguacuuuaguuaucgaugguguuacaccaaauaugauugauuacuuuggccguccuuacgaggguaucgcuguauucgacgguaaaaaaauuacaguuacugguacacuuuggaaugguaacaaaauuaucgacgaacguuugaucaauccagacgguucuuuauuauuccguguaacuaucaacggugugacugguuggcguuuaugugaacguaucuuagcauaaaccgguuuucccuuuuuuuuccucag

SEQ ID NO: 30 - DNA, 556 bp, Artificial Sequence
  Synthetic - NanoLuc-encoding artificial intron (plasmid)
gtgagtataccggtatggtattcactcttgaagacttcgtgggtgattggcgtcaaaccgcaggttacaacttagaccaggttttggagcagggtggtgtatcttctttatttcaaaaccttggtgtttcagtaacgccaattcaacgcatcgttttatcaggcgaaaacggccttaaaatcgatattcacgttatcatcccttacgagggcttgtcaggtgatcaaatgggccaaatcgaaaaaatctttaaagtagtatatccagttgatgatcaccactttaaagttatcttacattacggtactttagttatcgatggtgttacaccaaatatgattgattactttggccgtccttacgagggtatcgctgtattcgacggtaaaaaaattacagttactggtacactttggaatggtaacaaaattatcgacgaacgtttgatcaatccagacggttctttattattccgtgtaactatcaacggtgtgactggttggcgtttatgtgaacgtatcttagcataaaccggttttcccttttttttcctcag

SEQ ID NO: 31 - RNA, 556 nt, Artificial Sequence
  Synthetic - NanoLuc-encoding artificial intron (viral segment)
cugaggaaaaaaaagggaaaaccgguuuaugcuaagauacguucacauaaacgccaaccagucacaccguugauaguuacacggaauaauaaagaaccgucuggauugaucaaacguucgucgauaauuuuguuaccauuccaaaguguaccaguaacuguaauuuuuuuaccgucgaauacagcgauacccucguaaggacggccaaaguaaucaaucauauuugguguaacaccaucgauaacuaaaguaccguaauguaagauaacuuuaaaguggugaucaucaacuggauauacuacuuuaaagauuuuuucgauuuggcccauuugaucaccugacaagcccucguaagggaugauaacgugaauaucgauuuuaaggccguuuucgccugauaaaacgaugcguugaauuggcguuacugaaacaccaagguuuugaaauaaagaagauacaccacccugcuccaaaaccuggucuaaguuguaaccugcgguuugacgccaaucacccacgaagucuucaagagugaauaccauaccgguauacucac

SEQ ID NO: 32 - RNA, 472 nt, Artificial Sequence
  Synthetic - NEP-encoding artificial intron (mRNA)
gugaguauaccgguggaagcggugcuacuaauuuuucacuucucaaacaagcaggcgauguggaagaaaaccccggaccaauggacccaaauacagucucaucauuccaagauauccugcugaggauguccaaaaugcaacucgaaucgagcaguggggauuugaauggcaugauuacacaauuugaaagccugaagcuuuauagagacucccuuggggaagccgugaugagaaugggggaucuccacucucuacagaauaggaacgagaaguggcgcgaacaacugggccagaaauucgaagagauaagauggcuuauugaagaggugcgacauaaacugaaaauuacagagaauucauuugagcagaucacuuucaugcaagcacuacacuuacuucuagaaguggaacaggaaauuagaacuuuuagcuuucaauuaauauaaaccgguuuucccuuuuuuuuccucag

SEQ ID NO: 33 - DNA, 472 bp, Artificial Sequence
  Synthetic - NEP-encoding artificial intron (plasmid)
gtgagtataccggtggaagcggtgctactaatttttcacttctcaaacaagcaggcgatgtggaagaaaaccccggaccaatggacccaaatacagtctcatcattccaagatatcctgctgaggatgtccaaaatgcaactcgaatcgagcagtggggatttgaatggcatgattacacaatttgaaagcctgaagctttatagagactcccttggggaagccgtgatgagaatgggggatctccactctctacagaataggaacgagaagtggcgcgaacaactgggccagaaattcgaagagataagatggcttattgaagaggtgcgacataaactgaaaattacagagaattcatttgagcagatcactttcatgcaagcactacacttacttctagaagtggaacaggaaattagaacttttagctttcaattaatataaaccggttttcccttttttttcctcag

SEQ ID NO: 34 - RNA, 472 nt, Artificial Sequence
  Synthetic - NEP-encoding artificial intron (viral segment)
cugaggaaaaaaaagggaaaaccgguuuauauuaauugaaagcuaaaaguucuaauuuccuguuccacuucuagaaguaaguguagugcuugcaugaaagugaucugcucaaaugaauucucuguaauuuucaguuuaugucgcaccucuucaauaagccaucuuaucucuucgaauuucuggcccaguuguucgcgccacuucucguuccuauucuguagagaguggagaucccccauucucaucacggcuuccccaagggagucucuauaaagcuucaggcuuucaaauuguguaaucaugccauucaaauccccacugcucgauucgaguugcauuuuggacauccucagcaggauaucuuggaaugaugagacuguauuuggguccauugguccgggguuuucuuccacaucgccugcuuguuugagaagugaaaaauuaguagcaccgcuuccaccgguauacucac

SEQ ID NO: 35 - DNA, 131 bp, Artificial Sequence
  Synthetic - constitutively spliced artificial intron with splicing environment (FIG. 15)
aaggtgagtataccggtctctaaaagcgggcatgacttctagagtagtccagggtttccgagggtttccgtcgactcagctcgtctcgagggcgtactaactaccggttttcccttttttttcctcaggtg

SEQ ID NO: 36 - DNA, 562 bp, Artificial Sequence
  Synthetic - NanoLuc-encoding artificial intron with splicing environment (FIG. 16)
aaggtgagtataccggtatggtattcactcttgaagacttcgtgggtgattggcgtcaaaccgcaggttacaacttagaccaggttttggagcagggtggtgtatcttctttatttcaaaaccttggtgtttcagtaacgccaattcaacgcatcgttttatcaggcgaaaacggccttaaaatcgatattcacgttatcatcccttacgagggcttgtcaggtgatcaaatgggccaaatcgaaaaaatctttaaagtagtatatccagttgatgatcaccactttaaagttatcttacattacggtactttagttatcgatggtgttacaccaaatatgattgattactttggccgtccttacgagggtatcgctgtattcgacggtaaaaaaattacagttactggtacactttggaatggtaacaaaattatcgacgaacgtttgatcaatccagacggttctttattattccgtgtaactatcaacggtgtgactggttggcgtttatgtgaacgtatcttagcataaaccggttttcccttttttttcctcaggtg

SEQ ID NO: 37 - DNA, 1885 bp, Artificial Sequence
  Synthetic - Segment 6 of the PR8 NA-intron-NEP virus (FIG. 18)
agcgaaagcaggggtttaaaatgaatccaaatcagaaaataacaaccattggatcaatctgtctggtagtcggactaattagcctaatattgcaaatagggaatataatctcaatatggattagccattcaattcaaactggaagtcaaaaccatactggaatatgcaaccaaaacatcattacctataaaaatagcacctgggtaaaggacacaacttcagtgatattaaccggcaattcatctctttgtcccatccgtgggtgggctatatacagcaaagacaatagcataagaattggttccaaaggagacgtttttgtcataagagagccctttatttcatgttctcacttggaatgcaggaccttttttctgacccaaggtgagtataccggtggaagcggtgctactaatttttcacttctcaaacaagcaggcgatgtggaagaaaaccccggaccaatggacccaaatacagtctcatcattccaagatatcctgctgaggatgtccaaaatgcaactcgaatcgagcagtggggatttgaatggcatgattacacaatttgaaagcctgaagctttatagagactcccttggggaagccgtgatgagaatgggggatctccactctctacagaataggaacgagaagtggcgcgaacaactgggccagaaattcgaagagataagatggcttattgaagaggtgcgacataaactgaaaattacagagaattcatttgagcagatcactttcatgcaagcactacacttacttctagaagtggaacaggaaattagaacttttagctttcaattaatataaaccggttttcccttttttttcctcaggtgccttactgaatgacaagcattcaaatgggactgttaaggacagaagcccttatagggccttaatgagctgccctgtcggtgaagctccgtccccgtacaattcaagatttgaatcggttgcttggtcagcaagtgcatgtcatgatggcatgggctggctaacaatcggaatttcaggtccagataatggagcagtggctgtattaaaatacaacggcataataactgaaaccataaaaagttggaggaagaaaatattgaggacacaagagtctgaatgtgcctgtgtaaatggttcatgttttactataatgactgatggcccgagtgatgggctggcctcgtacaaaattttcaagatcgaaaaggggaaggttactaaatcaatagagttgaatgcacctaattctcactatgaggaatgttcctgttaccctgataccggcaaagtgatgtgtgtgtgcagagacaactggcatggttcgaaccggccatgggtgtctttcgatcaaaacctggattatcaaataggatacatctgcagtggggttttcggtgacaacccgcgtcccgaagatggaacaggcagctgtggtccagtgtatgttgatggagcaaacggagtaaagggattttcatataggtatggtaatggtgtttggataggaaggaccaaaagtcacagttccagacatgggtttgagatgatttgggatcctaatggatggacagagactgatagtaagttctctgttaggcaagatgttgtggcaatgactgattggtcagggtatagcggaagtttcgttcaacatcctgagctaacagggctagactgtatgaggccgtgcttctgggttgaattaatcaggggacgacctaaagaaaaaacaatctggactagtgcgagcagcatttctttttgtggcgtgaatagtgatactgtagattggtcttggccagacggtgctgagttgccattcagcattgacaagtagtctgttcaaaaaactccttgtttctact

SEQ ID NO: 38 - DNA, 890 bp, Artificial Sequence
  Synthetic - Segment 8 of the PR8 NA-intron-NEP virus (FIG. 19)
agcaaaagcagggtgacaaagacataatggatccaaacactgtgtcaagctttcaagtagattgctttctttggcatgtccgcaaacgagttgcagaccaagaactaggtgatgccccattccttgatcggcttcgccgagatcagaaatccctaagaggaaggggcagcaccctcggtctggacatcgagacagccacacgtgctggaaagcagatagtggagcggattctgaaagaagaatccgatgaggcacttaaaatgaccatggcctctgtacctgcgtcgcgttacctaactgacatgactcttgaggaaatgtcaagggactggtccatgctcatacccaagcagaaagtggcaggccctctttgtatcagaatggaccaggcgatcatggataagaacatcatactgaaagcgaacttcagtgtgatttttgaccggctggagactctaatattgctaagggctttcaccgaagagggagcaattgttggcgaaatttcaccattgccttctcttcctggacatactgctgaggatgtgaaaaatgcagttggagtcctcatcgggggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaataaagatgattgatttaagaagtgtgacactaactgtagataacatagaatagtttttagcaaataacatttatgtaagccttacatctattgcttgaagtggagcaagagataagaactttctcgtttcagcttatttaataataaaaaacacccttgtttctact

SEQ ID NO: 39 - DNA, 1906 bp, Artificial Sequence
  Synthetic - Segment 6 of the A/Hawaii/70/2019-NA-intron-NEP virus (FIG. 20)
agtttaaaatgaatccaaaccaaaagataataaccattggctctatctgtatgacaattggaatggctaacttaatattacaaattggaaacataatctcaatatgggttagccactcaattcaaattggaaatcaaagccagattgaaacatgcaataaaaacgtcatcacttatgaaaacaacacttgggtaaatcagacatatgttaacatcagcaacaccaactctgctgctagacagtcagtggcttccgtgaaattagcgggcaattcctctctctgccctgtcagtggatgggctatatacagtaaagacaacagtgtaagaatcggttccaaaggtgagtataccggtggaagcggtgctactaatttttcacttctcaaacaagcaggcgatgtggaagaaaaccccggaccaatggacccaaatacagtctcatcattccaagatatcctgctgaggatgtccaaaatgcaactcgaatcgagcagtggggatttgaatggcatgattacacaatttgaaagcctgaagctttatagagactcccttggggaagccgtgatgagaatgggggatctccactctctacagaataggaacgagaagtggcgcgaacaactgggccagaaattcgaagagataagatggcttattgaagaggtgcgacataaactgaaaattacagagaattcatttgagcagatcactttcatgcaagcactacacttacttctagaagtggaacaggaaattagaacttttagctttcaattaatataaaccggttttcccttttttttcctcaggtgatgtgtttgtcataagggaaccattcatatcatgctctcccttggaatgcagaaccttcttcttgactcaaggggctttgctaaatgacaaacactccaatggaaccattaaagacagaagcccatatcgaaccctaatgagctgtcctattggtgaagttccctctccatacaactcaagatttgagtcagtcgcttggtcagcaagtgcttgtcatgatggcaccaattggctaacaattggaatttctggcccagacagtggggcagtggctgtgttaaaatacaatggcataataacagacactatcaagagttggaggaacaatatattgagaacacaagagtctgaatgtgcatgtgtaaatggttcttgctttaccataatgaccgatggaccaagtgatggacaggcctcatacaaaatcttcagaatagaaaagggaaagataatcaaatcagtcgaaatgaaagcccctaattatcactatgaggaatgctcctgttaccctgattctagtgaaatcacatgtgtttgcagggataactggcatggctcgaatcgaccgtgggtgtctttcaaccagaatctggaataccagatgggatacatatgcagtggggttttcggagacaatccacgccctaatgataagacaggcagttgtggtccagtatcgtctaatggagcaaatggggtaaaaggattttcattcaaatacggcaatggtgtttggatagggagaactaagagcattagttcaagaaaaggttttgagatgatttgggatccgaatggatggactgggactgacaataaattctcaaaaaagcaagatatcgtaggaataaatgagtggtcagggtatagcgggagttttgttcagcatccagaactaacagggctgaattgtataagaccttgcttctgggttgaactaataagaggacgacccgaagagaacacaatctggactagcgggagcagcatatccttttgtggtgtagacagtgacattgtgggttggtcttggccagacggtgctgagttgccatttaccattgacaagtaatttgttcaaaaaaact

(19) United States
(12) Patent Application Publication
(10) Pub. No.: US 2024/0084267 A1
(43) Pub. Date: Mar. 14, 2024

(54) COMPOSITIONS AND METHODS FOR THE GENETIC MANIPULATION OF THE INFLUENZA VIRUS

(71) Applicant: Duke University, Durham, NC (US)

(72) Inventors: Nicholas HEATON, Durham, NC (US); Heather FROGGATT, Durham, NC (US); Kaitlyn BURKE, Durham, NC (US); Benjamin CHAMBERS, Durham, NC (US); Rebecca LEONARD, Durham, NC (US); Ryan CHAPARIAN, Durham, NC (US)

(21) Appl. No.: 18/271,983

(22) PCT Filed: Jan. 12, 2022
(86) PCT No.: PCT/US2022/012152

§ 371 Date: Jul. 12, 2023

Related U.S. Application Data
(60) Provisional application No. 63/136,296, filed on Jan. 12, 2021.

(51) Int. Cl. C12N 7/00; A61K 39/12; C12N 15/86

(52) U.S. Cl. CPC C12N 7/00; A61K 39/12; C12N 15/86; A61K 2039/5256; C12N 2760/16121; C12N 2760/16122; C12N 2760/16143; C12N 2760/16171

(57) ABSTRACT

The present invention provides recombinant viral segments comprising an artificial intron, DNA constructs encoding these viral segments, and recombinant viruses comprising these viral segments. Also provided are methods of making and using the recombinant viruses described herein.

### CROSS-REFERENCE TO RELATED APPLICATIONS

This application claims priority to U.S. Provisional Application No. 63/136,296 filed on Jan. 12, 2021, the contents of which are incorporated by reference in their entireties.

### STATEMENT REGARDING FEDERALLY SPONSORED RESEARCH

This invention was made with government support under grant number R01-HL142985 awarded by the National Institute for Allergy and Infectious Diseases, grant number R01-AI137031 awarded by the National Heart, Lung, and Blood Institute, and contract number 75N93019C00050 provided by the National Institute of Allergy and Infectious Diseases, National Institutes of Health. The government has certain rights in this invention.

### SEQUENCE LISTING

A Sequence Listing accompanies this application and is submitted as an ASCII text file of the sequence listing named “155554_00632_ST25.txt” which is 20,199 bytes in size and was created on Jan. 11, 2022. The sequence listing is electronically submitted via EFS-Web with the application and is incorporated herein by reference in its entirety.

### BACKGROUND

RNA viruses have a limited genetic space. To expand their coding capacity, many RNA viruses use alternative translation initiation sites and ribosomal frameshifting to access alternative reading frames encoding an additional protein or RNA product [1]. While most non-retroviral RNA viruses replicate in the cytoplasm, select others, such as viruses in the Orthomyxoviridae and Bornaviridae families, enter the nucleus to replicate their genomes and transcribe viral mRNAs [2]. Nuclear replication enables viral access to another tool for diversifying their encoded proteins: the host cell splicing machinery, which can allow distinct proteins to be produced from a single transcript.

Influenza A virus (IAV) uses the splicing of segments 7/M and 8/NS to generate multiple mRNA species and multiple proteins (M1/M2 and NS1/NEP, respectively) from a single viral segment [3,4]. Beyond generating multiple proteins from a single gene, viruses also take advantage of splicing to regulate viral gene expression. During IAV infections the ratio between two proteins produced from the NS segment, unspliced NS1, and spliced NEP, is skewed towards NS1 [5] to achieve the NS1 levels necessary to suppress host immune responses [6]. In contrast, as IAV infection progresses mRNAs produced from the M segment are spliced more often, increasing the amount of spliced M2 relative to unspliced M1 over time [7]. After contributing to viral entry, the M2 ion channel is thought to primarily be required late in replication during viral assembly [8]. Furthermore, splicing dysregulation in different host environments reduces viral replication efficiency, likely as a result of alterations to viral protein ratios [9-13]. These observations together demonstrate the importance of splicing in optimizing the influenza viral replication processes.

Despite the apparent tight controls of splicing, IAV segments 7 and 8 tolerate alterations to canonical splicing regulation. For example, in addition to M1/M2 splicing, there is also a third M segment-derived transcript, mRNA3, that is conserved but goes untranslated [14]. A limited number of strains also encode an additional 3′ splice site in NS that results in the NS3 transcript and protein [15]. Another group of strains encode an additional 5′ splice site in the M segment to produce the untranslated M42 transcript [16]. These findings show that additional splicing within already spliced IAV segments is tolerable and because these mutants occur naturally, potentially advantageous. Further, lab-generated viruses containing modified NS segments where splicing is eliminated and NS1 and NEP are “split” and separated by a 2A cleavage site are well tolerated and capable of encoding reporter proteins [17,18]. In contrast, analogous recombinant viruses “splitting” the M segment M1 and M2 sequences replicate poorly [19]. Nevertheless, recombinant IAVs that “split” both M and NS segments have been rescued, demonstrating that splicing can be eliminated from the IAV genome [19]. Thus, the importance and flexibility of splicing in IAV segments 7 and 8 are well recognized. However, the potential of splicing in additional IAV segments is generally less known.

Existing literature regarding genomic segment splicing across the Orthomyxoviridae family fails to reveal a clear consensus on the range or limits of viral RNA splicing. For instance, the shortest genome segments are frequently spliced in each member of the family: Segments 7 and 8 in 8-segmented IAV; segment 8 in 8-segmented influenza B virus (IBV); segments 6 and 7 in 7-segmented influenza C virus; segment 6 in 6-segmented Thogoto virus; and segment 7 in 7-segmented issavirus [20]. However, splicing in long segments has been reported as well. For example, a splicing product (PB2-S1) derived from the longest segment, segment 1/PB2, was identified in pre-2009 pandemic H1N1 IAVs [21]. Furthermore, viral genomes and transcripts are optimized during viral evolution, meaning that additional splicing is only observable when it confers an advantage; the range of segments where splicing is tolerable could differ significantly from where it is beneficial. Thus, it remains unclear if normally nonsplicing viral segments can tolerate splicing and what the effects on viral biology would be.

### SUMMARY

In a first aspect, the present invention provides recombinant viral segments comprising a viral segment from a negative-strand RNA virus (i.e., from the Orthomyxoviridae or Bornaviridae family) into which an artificial intron has been inserted. In these viral segments, the 3′ end of the artificial intron comprises the sequence AC and forms a 3′ splice site with the upstream portion of the viral segment, and the 5′ end of the artificial intron comprises the sequence CU and forms a 5′ splice site with the downstream portion of the viral segment. Additionally, the artificial intron comprises a branch site 20-50 bases downstream of the 5′ end.

In a second aspect, the present invention provides DNA constructs comprising the recombinant viral segments described herein.

In a third aspect, the present invention provides viruses comprising the recombinant viral segments described herein. The viruses are negative-strand RNA viruses from the Orthomyxoviridae or Bornaviridae family.

In a fourth aspect, the present invention provides methods of making a virus comprising an artificial intron. The methods comprise rescuing the virus with a DNA construct described herein.

In a fifth aspect, the present invention provides methods for using the recombinant viruses described herein. In a first embodiment, the recombinant viruses are used in a screening assay. In a second embodiment, the recombinant viruses are used to induce an immune response in a subject. In a third embodiment, the recombinant viruses are used to deliver a protein of interest to a cell in a subject.

### BRIEF DESCRIPTION OF THE DRAWINGS

FIG. 1 demonstrates that an artificial intron can be inserted into the IAV segment 6. (A) Diagram of the A/Puerto Rico/8/1934 H1N1 influenza A virus (IAV) segment 6/NA containing an intron and its protein products. ORF, open reading frame. (B) RT-PCR of WT PR8 virus (white) and PR8-NA-intron virus (red) segment 6/NA at passages 0 and 4 from serial PR8-NA-intron virus infections (MOI=0.01, multicycle, 72 h passages) on MDCK cells (representative of three independent experiments). (C) Growth kinetics of WT PR8 and PR8-NA-intron virus infections (MOI=0.001, multicycle) on MDCK cells, measured using HA assays (mean with SD, n=3 independent experiments). HAU, hemagglutination units; HPI, hours post-infection. (D) End point titers of WT PR8 and PR8-NA-intron virus infections (MOI=0.01, multicycle, 72 h) on MDCK cells, measured using plaque assays (mean with SD, n=3 independent experiments, unpaired Student's t-test). PFU, plaque forming units. (E) PR8 NA and NP RNA levels from mock, WT PR8, PR8-NA-intron virus infections (MOI=2, single cycle, 8 h) on MDCK cells, measured using one-step RT-qPCR (mean with SD, n=3 independent experiments, unpaired Student's t-test). Arbitrary units (a.u.) determined relative to 18S; ND, not determined. (F) Western blots for PR8 NA, NP, and PB1 proteins from mock, PR8 WT, and PR8-NA-intron virus infections (MOI=0.01, multicycle) on MDCK cells (representative of three independent experiments). GAPDH was used as a loading control. (G) PR8 segment 7/M and segment 8/NS mRNA splicing rates from WT PR8 virus infections (MOI=2, single cycle, 8 h) on MDCK cells, measured using two-step RT-qPCR (mean with SD, n=3 independent experiments). (H) PR8 segment 6/NA-intron mRNA splicing rates from PR8-NA-intron virus infections (MOI=2, single cycle, 8 h) on MDCK cells, measured using two-step RT-qPCR (mean with SD, n=3 independent experiments). (I) Growth kinetics of WT PR8 and PR8-NA-intron virus infections (100 PFU) in embryonated chicken eggs, measured using plaque assays (mean with SD, n=3 eggs per group, unpaired Student's t-test). For all panels: *P<0.05, **P<0.001, and ns=not significant.

FIG. 2 demonstrates that intronic length has minor effects on viral fitness. (A) Diagrams of PR8 IAV segment 6/NA with artificial introns of varying lengths (gray) compared to the initial sequence (red). (B) RT-PCR for segment 6/NA of WT PR8 virus and PR8-NA-intron (varying lengths) viruses. (C) Growth kinetics of WT PR8 and PR8-NA-intron (varying lengths) virus infections (MOI=0.001, multicycle) on MDCK cells, measured using HA assays (mean with SD, n=3 independent experiments). (D) End point titers of WT PR8 and PR8-NA-intron (varying lengths) virus infections (MOI=0.01, multicycle, 72 h) on MDCK cells, measured using plaque assays (mean with SD, n=3 independent experiments, one-way ANOVA). (E) PR8 segment 6/NA-intron mRNA splicing rates from PR8-NA-intron (varying lengths) virus infections (MOI=2, single cycle, 8 h) on MDCK cells, measured using two-step RT-qPCR (mean with SD, n=3 independent experiments).

FIG. 3 demonstrates that artificial introns can be used for expression of a reporter protein. (A) Diagram of the PR8 IAV segment 6/NA containing an intron encoding a NanoLuc (NL) reporter and its protein products. (B) RT-PCR of WT PR8 virus (white) and PR8-NA-intNL virus (blue) segment 6/NA. (C) Growth kinetics of WT PR8 and PR8-NA-intNL virus infections (MOI=0.001, multicycle) on MDCK cells, measured using HA assays (mean with SD, n=3 independent experiments). (D) End point titers of WT PR8 and PR8-NA-intNL virus infections (MOI=0.01, multicycle, 72 h) on MDCK cells, measured using plaque assays (mean with SD, n=3 independent experiments, unpaired Student's t-test). (E) Western blots for PR8 NA, NP and PB1 proteins from mock, PR8 WT, and PR8-NA-intNL virus infections (MOI=0.01, multicycle) on MDCK cells (representative of three independent experiments). GAPDH was used as a loading control. (F) PR8 segment 6/NA-intNL mRNA splicing rates from PR8-NA-intNL virus infections (MOI=2, single cycle, 8 h) on MDCK cells, measured using two-step RT-qPCR (mean with SD, n=3 independent experiments). (G) Luciferase levels from PR8-NA-intNL virus infections at different MOIs (single cycle, 8 h) on MDCK cells (mean with SD, n=3 independent experiments, simple linear regression with goodness of fit). MOI, multiplicity of infection; RLU, relative light units. (H) Luciferase levels from WT PR8 and PR8-NA-intNL single cycle virus infections (MOI=0.1, single cycle) on MDCK cells (mean with SD, n=3 independent experiments, unpaired Student's t-test relative to WT). Gray shading indicates infection incubation period. (I) Luciferase levels from WT PR8 and PR8-NA-intNL multicycle virus infections (MOI=0.001, multicycle) on MDCK cells (mean with SD, n=3 independent experiments, unpaired Student's t-test relative to WT). (J) Luciferase levels from infections (MOI=0.001, multicycle, 24 h) on MDCK cells using PR8-NA-intNL virus (passage 0) and supernatants from serial PR8-NA-intNL virus infections (MOI=0.001, multicycle, 72 h passages) on MDCK cells (mean with SD, n=3 independent experiments, one-way ANOVA). For all panels: *P<0.05, **P<0.001 and ns=not significant.

FIG. 4 demonstrates that IAVs harboring introns with luciferase genes can be used to evaluate antibodies and antiviral compounds. (A) Virus levels from Baloxavir treated WT PR8 (left) and PR8-NA-intNL (middle) virus infections (MOI=0.05, multicycle, 48 h) measured using HA assays and luciferase levels from Baloxavir treated PR8-NA-intNL (right) virus infections (MOI=0.01, multicycle, 24 h) on MDCK cells (mean with SD, n=3 independent experiments, nonlinear regression dose-response curve). Normalized to vehicle-treated, infected controls. (B) Virus levels from PY102 anti-H1 antibody pre-incubated WT PR8 (left) and PR8-NA-intNL (middle) virus infections (40 virions/well) measured using PRNTs and luciferase levels from PY102 anti-PR8 HA antibody pre-incubated PR8-NA-intNL (right) virus infections (MOI=0.001, multicycle, 24 h) on MDCK cells (mean with SD, n=3 independent experiments, nonlinear regression dose-response curve). Normalized to PBS pre-incubated controls. PRNTs, plaque reduction neutralization tests. (C) Virus levels from anti-PR8 mouse sera pre-incubated WT PR8 (left) and PR8-NA-intNL (middle) virus infections (40 virions/well) measured using PRNTs and luciferase levels from anti-PR8 mouse sera pre-incubated PR8-NA-intNL (right) virus infections (MOI=0.001, multicycle, 24 h) on MDCK cells (mean with SD, n=3 independent experiments, nonlinear regression dose-response curve). Normalized to PBS pre-incubated controls; rec.dil., reciprocal dilution.

FIG. 5 demonstrates that IAVs harboring introns with luciferase genes cause disease in a mouse model. (A) Relative bodyweights of WT PR8 (left) and PR8-NA-intNL (right) virus infected C57BL/6J mice (age matched, 6-12 weeks, female) (mean with SD, n=5 mice per group). DPI, days post-infection. (B) Survival of WT PR8 (left) and PR8-NA-intNL (right) virus infected BL/6 mice (n=5 mice per group). (C) Lung viral titers from WT PR8 and PR8-NA-intNL virus infected (100 PFU) BL/6 mice, measured using plaque assays (mean with SD, n=5 mice per group, unpaired Student's t-test relative to WT). (D) PR8 segment 6/NA-intNL mRNA splicing rates from PR8-NA-intNL virus infected (100 PFU, 4 d) BL/6 mouse lungs, measured using two-step RT-qPCR (mean with SD, n=5 mice per group). (E) Luciferase levels from WT PR8 and PR8-NA-intNL virus infected (100 PFU) BL/6 mice lungs (mean with SD, n=5 mice per group, unpaired Student's t-test relative to WT). (F) Luciferase and PR8 NP RNA levels from PR8-NA-intNL virus infected (10 PFU) BL/6 mouse lungs (mean with SD, n=3 mice per group, Pearson correlation coefficient). Arbitrary units (a.u.) determined relative to 18S. Background levels of detection from uninfected mice are shown at 0 DPI. (G) RT-PCR of WT PR8 virus (white) and PR8-NA-intNL virus (blue) segment 6/NA passages 0 to 4 from serial infections (1000 PFU, 3 d passages) in BL/6 mouse lungs (representative of three independent experiments). For all panels: *P<0.05, **P<0.001 and ns=not significant.

FIG. 6 demonstrates that intronic reporters in PR8 segment 5 allow generation of 6+2 reassortment viral reporter strains. (A) Diagram of the PR8 IAV segment 5/NP containing an intron encoding a NanoLuc reporter and its protein products. (B) RT-PCR of WT PR8 virus (yellow) and PR8-NP-intNL virus (green) segment 5/NP. (C) Growth kinetics of WT PR8 and PR8-NP-intNL virus infections (MOI=0.001, multicycle) on MDCK cells, measured using HA assays (mean with SD, n=3 independent experiments). (D) End point titers of WT PR8 and PR8-NP-intNL virus infections (MOI=0.01, multicycle, 72 h) on MDCK cells, measured using plaque assays (mean with SD, n=3 independent experiments, unpaired Student's t-test). (E) Western blots for PR8 NA, NP and PB1 proteins from mock, PR8 WT, and PR8-NP-intNL virus infections (MOI=0.01, multicycle) on MDCK cells (representative of three independent experiments). GAPDH was used as a loading control. (F) PR8 segment 5/NP-intNL mRNA splicing rates from PR8-NP-intNL virus infections (MOI=2, single cycle, 8 h) on MDCK cells, measured using two-step RT-qPCR (mean with SD, n=3 independent experiments). (G) Luciferase levels from PR8-NP-intNL virus infections at different MOIs (single cycle, 8 h) on MDCK cells (mean with SD, n=3 independent experiments, simple linear regression with goodness of fit). (H) Diagram of the 6+2 influenza reassortant with SW/HN/SN13/18 (SW18) glycoproteins on a PR8-NP-intNL background. (I) Growth kinetics of WT PR8, PR8-NP-intNL, and 6+2 SW18 virus infections (MOI=0.001, multicycle) on MDCK cells, measured using HA assays (mean with SD, n=3 independent experiments). The downward error bar for the WT PR8 24 h data point could not be plotted on a log scale. (J) End point titers of WT PR8, PR8-NP-intNL, and 6+2 SW18 virus infections (MOI=0.001, multicycle, 72 h) on MDCK cells, measured using plaque assays (mean with SD, n=3 independent experiments, unpaired Student's t-test). (K) Luciferase levels from mock, PR8-NP-intNL, and 6+2 SW18 virus infections (MOI=0.001, 24 h, multicycle) on MDCK cells (mean with SD, n=3 independent experiments, unpaired Student's t-test). For all panels: *P<0.05, **P<0.001 and ns=not significant.

FIG. 7 demonstrates that artificial intron insertion can be used to develop reporter strains in different viral genetic backgrounds. (A) Diagram demonstrating how to introduce an artificial intron encoding a reporter into non-splicing, non-frameshifting IAV segments using A/Wyoming/03/2003 (H3N2) segment 6/NA as an example. (B) RT-PCR of WT Wyo/03 virus (white) and Wyo/03-NA-intNL virus (blue) segment 6/NA. (C) End point titers of WT Wyo/03 and Wyo/03-NA-intNL virus infections (MOI=0.0001, multicycle, 72 h) on MDCK cells, measured using plaque assays (mean with SD, n=4 independent experiments, unpaired Student's t-test). (D) Luciferase levels from WT Wyo/03 and Wyo/03-NA-intNL virus infections (MOI=0.0001, multicycle, 24 h) on MDCK cells (mean with SD, n=3 independent experiments, unpaired Student's t-test). For all panels: *P<0.05, **P<0.001 and ns=not significant.

FIG. 8 illustrates the two-step RT-qPCR assay that was used to determine PR8 mRNA splicing rates. (A) Diagram of two-step RT-qPCR used to determine splicing rates. First, RNA was collected from infected cells/tissue and reverse-transcribed into DNA using oligo(dT) primers to select for mRNAs. Next, SYBR Green-based qPCR was performed using primers that targeted either 1) all mRNAs derived from an IAV segment, or 2) specifically unspliced mRNAs from that segment. Absolute values for the mRNA transcript copy numbers were determined using a standard curve of known plasmid concentrations encoding the segment of interest. Finally, using the generated standard curve, transcript copy numbers were determined for both 1) all mRNAs and 2) unspliced (un) mRNAs derived from the segment of interest and used to determine what percent of all transcripts from one segment were spliced. (B) Top: Diagram of “M1/2” (striped) and “M1 only” (solid) dye-based qPCR primer locations on PR8 M mRNAs. Bottom, left: Absolute standard curve detecting “M1/2” and “M1 only” sequences from a plasmid containing the PR8 M segment. Bottom, right: Absolute standard curve detecting “M1/2” and “M1 only” sequences from a plasmid containing the PR8 M2 ORF. UTR, untranslated region. (C) Top: Diagram of “NS1/NEP” (striped) and “NS1 only” (solid) dye-based qPCR primer locations on PR8 NS mRNAs. Bottom, right: Absolute standard curve detecting “NS1/NEP” and “NS1 only” sequences from a plasmid containing the PR8 NS segment. Bottom, left: Absolute standard curve detecting “NS1/NEP” and “NS1 only” sequences from a plasmid containing the PR8 NEP ORF. (D) Top: Diagram of “NA-int/NA” (white) and “NA-int only” (red) dye-based qPCR primer locations on PR8 NA-intron mRNAs. Bottom, left: Absolute standard curve detecting “NA-int/NA” and “NA-int only” sequences from a plasmid containing the PR8 NA intron-containing segment. Bottom, right: Absolute standard curve detecting “NA-int/NA” and “NA-int only” sequences from a plasmid containing the PR8 NA segment.

FIG. 9 demonstrates that artificial intron splicing rates are dependent on cellular environments. (A) PR8 segment 6/NA-intron mRNA splicing rates over time from PR8-NA-intron virus infections (MOI=2, single cycle) on MDCK cells, measured using two-step RT-qPCR (mean with SD, n=3 independent experiments). (B) PR8 NP RNA levels from PR8-NA-intron virus infections (MOI=2, single cycle, 8 h) on human lung A549 and avian embryonic DF-1 cells, measured using one-step RT-qPCR (mean with SD, n=3 independent experiments, unpaired Student's t-test). Arbitrary units (a.u.) determined relative to 18S. (C) PR8 segment 6/NA-intron mRNA splicing rates during PR8-NA-intron virus infections (MOI=2, single cycle, 8 h) on human lung A549 and avian embryonic DF-1 cells, measured using two-step RT-qPCR (mean with SD, n=3 independent experiments, unpaired Student's t-test). For all panels: *P<0.05, **P<0.001 and ns=not significant.

FIG. 10 demonstrates that an intronic reporter sequence in PR8 segment 6 is maintained during virus propagation in cell culture. (A) RT-PCR of WT PR8 virus (white) and PR8-NA-intNL virus (blue) segment 6/NA passages 0 to 10 from serial PR8-NA-intNL virus infections (MOI=0.001, multicycle, 72 h passages) on MDCK cells (representative of three independent experiments). #After passage 10, viruses from infection supernatants were plaque purified and the intron-containing viral genomic segment was sequenced via Sanger sequencing. In all cases the intron and NanoLuc gene were present; however, within the artificial intron we detected ≤3 nucleotide deletions or mismatches in the 3′ region of the intron at the ends of homopolymeric runs. This could either be the result of selection for mutant intron sequences or limitations of the sequencing itself (B) Schematic depiction of the A549 cell passaging experiments. (C) Top: Luciferase levels from infections (multicycle, 24 h) on A549 cells using PR8-NA-intNL virus (passage 0) and supernatants from serial PR8-NA-intNL virus infections (MOI=0.5, multicycle, 72 h passages) on A549 cells (mean with SD, n=3 independent experiments). Bottom: RT-PCR of WT PR8 virus (white) and PR8-NA-intNL virus (blue) virus from passages 0 to 4 from serial PR8-NA-intNL virus infections (MOI=0.5, multicycle, 72 h passages) on A549 cells (representative of three independent experiments).

FIG. 11 demonstrates that embryonated chicken eggs are not optimal for repeated propagation of intronic reporter influenza viruses. (A) Growth kinetics of WT PR8 and PR8-NA-intNL virus infections (100 PFU) in embryonated chicken eggs, measured using plaque assays (mean with SD, n=3 eggs per group, unpaired Student's t-test relative to WT). (B) Luciferase levels in egg allantoic fluid from WT PR8 and PR8-NA-intNL virus infections (100 PFU) in embryonated chicken eggs (mean with SD, n=3 eggs per group, unpaired Student's t-test relative to WT). (C) Schematic depiction of the egg passaging experiments. (D) Titers in egg allantoic fluid from serial PR8-NA-intNL virus infections (100 PFU, 72 h passages) in embryonated chicken eggs, measured using plaque assays (mean with SD, n=3 eggs per group). (E) Luciferase levels in egg allantoic fluid from serial PR8-NA-intNL virus infections (100 PFU, 72 h passages) in embryonated chicken eggs (mean with SD, n=3 eggs per group); un, uninfected. (F) RT-PCR of WT PR8 virus (white) and PR8-NA-intNL virus (blue) segment 6/NA from egg passages 0 to 4; exp, independent experiment. #After passage 4, viruses from infected egg allantoic fluid were plaque purified and the intron-containing viral genomic segment was sequenced via Sanger sequencing. In all cases we detected a mixed population within one stock, with some apparently wild-type revertant viruses without any residual intron sequence, and some viruses where the intron and NanoLuc gene were present; however, within the artificial intron we detected ≤3 nucleotide deletions or mismatches in the 3′ region of the intron at the ends of homopolymeric runs. This could either be the result of selection for mutant intron sequences or limitations of the sequencing itself. For all panels: *P<0.05, **P<0.001 and ns=not significant.

FIG. 12 confirms PR8-NA-intNL virus stability during mouse infections. (A) Schematic depiction of mouse passaging experiments. (B) Luciferase levels in mouse lung homogenates from serial PR8-NA-intNL virus infections (1000 PFU, 3 d passages) in BL/6 mice (mean with SD, n=3 mice per group); un, uninfected. (C) Titers in mouse lung homogenates from serial PR8-NA-intNL virus infections (1000 PFU, 3 d passages) in BL/6 mice (mean with SD, n=3 mice per group). (D) Luciferase levels from infections (MOI=0.001, multicycle, 24 h) on MDCK cells using PR8-NA-intNL virus (passage 0) and mouse lung homogenates from serial PR8-NA-intNL virus infections (1000 PFU, 3 d passages) in BL/6 mice (mean with SD, n=3 mice per group).

FIG. 13 illustrates how PR8-NP-intNL mRNA splicing rates and virus stability are determined in cell culture. (A) Top: Schematic depiction of “NP-int/NP” (yellow) and “NP-int only” (green) dye-based qPCR primer locations on PR8 NP-intNL mRNAs. Bottom, left: Absolute standard curve detecting “NP-int/NP” and “NP-int only” sequences from a plasmid containing an PR8 NP intron-containing segment. Bottom, right: Absolute standard curve detecting “NP-int/NP” and “NP-int only” sequences from a plasmid containing an PR8 NP segment. (B) Top: Luciferase levels from infections (MOI=0.001, multicycle, 24 h) on MDCK cells using PR8-NP-intNL virus (passage 0) and supernatants from serial PR8-NP-intNL virus infections (MOI=0.001, multicycle, 72 h passages) on MDCK cells (mean with SD, n=3 independent experiments, one-way ANOVA with Dunnett's multiple comparisons test relative to passage 0). Bottom: RT-PCR of WT PR8 virus (yellow) and PR8-NP-intNL virus (green) segment 5/NP passages 0 to 10 from serial PR8-NP-intNL virus infections (MOI=0.001, multicycle, 72 h passages) on MDCK cells (representative of three independent experiments). #After passage 10, viruses from infection supernatants were plaque purified and the intron-containing viral genomic segment was sequenced via Sanger sequencing. In all cases we detected apparently wild-type revertant viruses that did not harbor any residual intron sequence. For all panels: *P<0.05, **P<0.001 and ns=not significant.

FIG. 14 shows growth kinetics of WT Wyo/03 and Wyo/03-NA-intNL virus infections (MOI=0.0001, multicycle) on MDCK cells, measured using HA assays (mean with SD, n=3 independent experiments). The downward error bar for the NA-intNL 72 hour data point could not be plotted on a log scale.

FIG. 15 shows the sequence (SEQ ID NO: 35) of the constitutively spliced artificial intron that was tested in Example 1.

FIG. 16 shows the sequence (SEQ ID NO: 36) of the NanoLuc-encoding artificial intron that was tested in Example 1.

FIG. 17 shows the full, unprocessed western blot exposures corresponding to the composite images shown in FIGS. 1, 3 and 6. (A) Uncropped western blots from FIG. 1F. (B) Uncropped western blots from FIG. 3E. (C) Uncropped western blots from FIG. 6E. For all panels: red box, cropping in figure panel; white arrow, membrane cut.

FIG. 18 shows the genetic organization of segment 6 cDNA of the PR8 NA-intron-NEP virus. (Top) Schematic depiction of segment 6 (which natively encodes the NA gene) in the PR8-NA-intron-NEP virus. (Bottom) Nucleotide sequence of the segment (SEQ ID NO: 37). The genetic features are color-matched in the schematic and the sequence.

FIG. 19 shows the genetic organization of segment 8 cDNA of the PR8 NA-intron-NEP virus. (Top) Schematic depiction of segment 8 (which natively encodes the NS1/NEP genes) in the PR8-NA-intron-NEP virus. (Bottom) Nucleotide sequence of the segment (SEQ ID NO: 38). The genetic features are color-matched in the schematic and the sequence.

FIG. 20 shows the genetic organization of segment 6 cDNA of the A/Hawaii/70/2019-NA-intron-NEP virus. (Top) Schematic depiction of segment 6 (which natively encodes the NA gene) in the A/Hawaii/70/2019-NA-intron-NEP virus. (Bottom) Nucleotide sequence of the segment (SEQ ID NO: 39). The genetic features are color-matched in the schematic and the sequence.

FIG. 21 shows the lethality of PR8-NA-intron-NEP virus in C57BL/6J mice. C57BL/6J mice were infected intranasally with either WT PR8 (left) or attenuated PR8-NA-intron-NEP (right) virus at varying doses. Bodyweights were monitored and recorded for 14 days post infection. The dotted line represents the humane bodyweight cutoff (75% of starting weight). PFU=plaque forming unit.

FIG. 22 demonstrates that HA/NA immune responses were mounted against the PR8-NA-intron-NEP live-attenuated virus in infected mice. Sera was collected from C57BL/6J mice that were inoculated with either 10 PFU WT PR8 virus or 1,000 PFU PR8-NA-intron-NEP attenuated virus. ELISAs against soluble PR8 HA protein, soluble PR8 NA protein, and whole PR8 virus were performed to quantify the immune responses to each virus.

### DETAILED DESCRIPTION

The present invention provides recombinant viral segments comprising an artificial intron, DNA constructs encoding these viral segments, and recombinant viruses comprising these viral segments. Also provided are methods of making and using the recombinant viruses described herein.

To experimentally probe the influenza viral genome for tolerance of additional splicing, the present inventors designed artificial introns with different characteristics and inserted them into the otherwise nonsplicing segments of the influenza A virus (IAV) genome. As is demonstrated in Example 1, viruses containing artificial introns were viable, and the composition of the intron itself was not a major constraint on the tolerance of artificially introduced splicing. In fact, introns harboring a full-length reporter gene were well tolerated and could be used to express functional reporter protein from unspliced transcripts. One advantage of introducing reporter genes into a viral genome in this manner is that it requires limited manipulation of the viral RNA (e.g., no packaging signal mapping is required). Based on these experiments, which were performed in a laboratory adapted H1N1 IAV genetic background, the inventors developed a set of rules for the insertion of artificial introns into any IAV genome, and they then demonstrated the utility of this approach by generating an intronic reporter in the H3N2 IAV genetic background. In Example 2, the inventors generated IAV particles comprising an artificial intron encoding a IAV protein that is normally expressed from a different viral segment.

Surprisingly, they found that this recombinant virus produced a more robust immune response than its wild-type counterpart, suggesting that artificial introns could potentially be used to produce superior vaccine platforms.

### Recombinant Viral Segments:

In a first aspect, the present invention provides recombinant viral segments comprising a viral segment from a negative-strand RNA virus (i.e., from the Orthomyxoviridae or Bornaviridae family) into which an artificial intron has been inserted. In these viral segments, the 3′ end of the artificial intron comprises the sequence AC and forms a 3′ splice site with the upstream portion of the viral segment, and the 5′ end of the artificial intron comprises the sequence CU and forms a 5′ splice site with the downstream portion of the viral segment. Additionally, the artificial intron comprises a branch site 20-50 bases downstream of the 5′ end.

The genomes of RNA viruses are commonly divided into multiple distinct RNA molecules, referred to as “viral segments”. For example, the genomes of influenza A viruses contain eight segments of single-stranded RNA that each encode 1-2 proteins. Specifically, segment 1 encodes polymerase basic protein 2 (PB2), segment 2 encodes polymerase basic protein 1 (PB1), segment 3 encodes polymerase acidic protein (PA), segment 4 encodes hemagglutinin (HA), segment 5 encodes nucleoprotein (NP), segment 6 encodes neuraminidase (NA), segment 7 encodes matrix protein 1 (M1) and matrix protein 2 (M2), and segment 8 encodes non-structural protein 1 (NS1) and non-structural protein 2 (NS2; also referred to as NEP).

As used herein, the term “recombinant viral segment” refers to an artificially constructed viral segment that includes at least one heterologous RNA sequence that is not natively found in the viral segment. The recombinant viral segments of the present invention comprise a viral segment into which an artificial intron has been inserted. The generation of recombinant polynucleotides can be accomplished using standard techniques (e.g., cloning, DNA and RNA isolation, amplification, and purification) that are well known in the art.

The term “artificial intron” refers to an intron (i.e., a nucleotide sequence within a gene that may be removed by RNA splicing during maturation of an mRNA) that was artificially introduced into a genome and is not naturally occurring. This term is used herein to refer to a sequence comprising negative-sense RNA, DNA, or positive-sense RNA. To produce a virus comprising a recombinant viral segment, the viral segment (which is negative-sense RNA) is transcribed from a DNA plasmid and assembled into a viral particle in a cell. Then, when the viral particle infects a host cell, mRNA (which is positive-sense RNA) is transcribed from the viral segment and is spliced by splicing machinery of the host cell. Thus, the version of the artificial intron sequence that is found in the viral segment is the reverse complement of the mRNA sequence that is ultimately spliced, and is the reverse complement of the DNA sequence that may have been used to generate (i.e., rescue) the virus. For clarity, all three of the sequences used in the artificial introns described herein are provided in Table 1.

TABLE 1
Conversion between forms of artificial intron sequences

| | Sequence in mRNA | Sequence in | |
|---|---|---|---|
| | synthesized | plasmid | Sequence in |
| | from the viral | encoding | viral segment |
| | segment or plasmid | the viral | (negative- |
| Component | (positive-sense RNA) | segment (DNA) | sense RNA) |
| 5′ splicing | MAG | MAG | CUK |
| environment | (e.g., AAG) | (e.g., AAG) | (e.g., CUU) |
| Splice | GU | GT | AC |
| donor | | | |
| 5′ end of | GURAGU | GTRAGT | ACUYAC |
| artificial | (e.g., GUGAGU) | (e.g., GTGAGT) | (e.g., ACUCAC) |
| intron | | | |
| Branch site | YURAY | YTRAY | RUYAR |
| | (e.g., CUAAC) | (e.g., CTAAC) | (e.g., GUUAG) |
| Splice | AG | AG | CU |
| acceptor | | | |
| 3′ end of | Y₁₃₋₂₀NCAG | Y₁₃₋₂₀NCAG | CUGNR₁₃₋₂₀ |
| artificial | (e.g., | (e.g., | (e.g., |
| intron | UUUUCCCUUUUUUU | TTTTCCCTTTTTTTT | CUGAGGAAAAAAA |
| | UCCUCAG (SEQ ID | CCTCAG (SEQ ID | AGGGAAAA |
| | NO: 23)) | NO: 24)) | (SEQ ID NO: 25)) |
| 3′ splicing | GNN | GNN | NNC |
| environment | (e.g., GUG) | (e.g., GTG) | (e.g., CAC) |
| Full-length | GUGAGUAUACCGGU | GTGAGTATACCGGT | CUGAGGAAAAAAA |
| constitutively | CUCUAAAAGCGGGC | CTCTAAAAGCGGGC | AGGGAAAACCGGU |
| spliced | AUGACUUCUAGAGU | ATGACTTCTAGAGT | AGUUAGUACGCCC |
| artificial | AGUCCAGGGUUUCC | AGTCCAGGGTTTCC | UCGAGACGAGCUG |
| intron | GAGGGUUUCCGUCG | GAGGGTTTCCGTCG | AGUCGACGGAAAC |
| | ACUCAGCUCGUCUC | ACTCAGCTCGTCTC | CCUCGGAAACCCU |
| | GAGGGCGUACUAAC | GAGGGCGTACTAAC | GGACUACUCUAGA |
| | UACCGGUUUUCCCU | TACCGGTTTTCCCTT | AGUCAUGCCCGCU |
| | UUUUUUUCCUCAG | TTTTTTCCTCAG | UUUAGAGACCGGU |
| | (SEQ ID NO: 26) | (SEQ ID NO: 27) | AUACUCAC |
| | | | (SEQ ID NO: 28) |
| Full-length | GUGAGUAUACCGGU | GTGAGTATACCGGT | CUGAGGAAAAAAA |
| NanoLuc- | AUGGUAUUCACUCU | ATGGTATTCACTCT | AGGGAAAACCGGU |
| encoding | UGAAGACUUCGUGG | TGAAGACTTCGTGG | UUAUGCUAAGAUA |
| artificial | GUGAUUGGCGUCAA | GTGATTGGCGTCAA | CGUUCACAUAAAC |
| intron | ACCGCAGGUUACAA | ACCGCAGGTTACAA | GCCAACCAGUCAC |
| | CUUAGACCAGGUUU | CTTAGACCAGGTTT | ACCGUUGAUAGUU |
| | UGGAGCAGGGUGG | TGGAGCAGGGTGGT | ACACGGAAUAAUA |
| | UGUAUCUUCUUUAU | GTATCTTCTTTATTT | AAGAACCGUCUGG |
| | UUCAAAACCUUGGU | CAAAACCTTGGTGT | AUUGAUCAAACGU |
| | GUUUCAGUAACGCC | TTCAGTAACGCCAA | UCGUCGAUAAUUU |
| | AAUUCAACGCAUCG | TTCAACGCATCGTT | UGUUACCAUUCCA |
| | UUUUAUCAGGCGAA | TTATCAGGCGAAAA | AAGUGUACCAGUA |
| | AACGGCCUUAAAAU | CGGCCTTAAAATCG | ACUGUAAUUUUUU |
| | CGAUAUUCACGUUA | ATATTCACGTTATC | UACCGUCGAAUAC |
| | UCAUCCCUUACGAG | ATCCCTTACGAGGG | AGCGAUACCCUCG |
| | GGCUUGUCAGGUGA | CTTGTCAGGTGATC | UAAGGACGGCCAA |
| | UCAAAUGGGCCAAA | AAATGGGCCAAATC | AGUAAUCAAUCAU |
| | UCGAAAAAAUCUUU | GAAAAAATCTTTAA | AUUUGGUGUAACA |
| | AAAGUAGUAUAUCC | AGTAGTATATCCAG | CCAUCGAUAACUA |
| | AGUUGAUGAUCACC | TTGATGATCACCAC | AAGUACCGUAAUG |
| | ACUUUAAAGUUAUC | TTTAAAGTTATCTT | UAAGAUAACUUUA |
| | UUACAUUACGGUAC | ACATTACGGTACTT | AAGUGGUGAUCAU |
| | UUUAGUUAUCGAU | TAGTTATCGATGGT | CAACUGGAUAUAC |
| | GGUGUUACACCAAA | GTTACACCAAATAT | UACUUUAAAGAUU |
| | UAUGAUUGAUUAC | GATTGATTACTTTG | UUUUCGAUUUGGC |
| | UUUGGCCGUCCUUA | GCCGTCCTTACGAG | CCAUUUGAUCACC |
| | CGAGGGUAUCGCUG | GGTATCGCTGTATT | UGACAAGCCCUCG |
| | UAUUCGACGGUAAA | CGACGGTAAAAAA | UAAGGGAUGAUAA |
| | AAAAUUACAGUUAC | ATTACAGTTACTGG | CGUGAAUAUCGAU |
| | UGGUACACUUUGGA | TACACTTTGGAATG | UUUAAGGCCGUUU |
| | AUGGUAACAAAAU | GTAACAAAATTATC | UCGCCUGAUAAAA |
| | UAUCGACGAACGUU | GACGAACGTTTGAT | CGAUGCGUUGAAU |
| | UGAUCAAUCCAGAC | CAATCCAGACGGTT | UGGCGUUACUGAA |
| | GGUUCUUUAUUAU | CTTTATTATTCCGTG | ACACCAAGGUUUU |
| | UCCGUGUAACUAUC | TAACTATCAACGGT | GAAAUAAAGAAGA |
| | AACGGUGUGACUGG | GTGACTGGTTGGCG | UACACCACCCUGCU |
| | UUGGCGUUUAUGU | TTTATGTGAACGTA | CCAAAACCUGGUC |
| | GAACGUAUCUUAGC | TCTTAGCATAAACC | UAAGUUGUAACCU |
| | AUAAACCGGUUUUC | GGTTTTCCCTTTTTT | GCGGUUUGACGCC |
| | CCUUUUUUUUCCUC | TTCCTCAG (SEQ ID | AAUCACCCACGAA |
| | AG (SEQ ID NO: 29) | NO: 30) | GUCUUCAAGAGUG |
| | | | AAUACCAUACCGG |
| | | | UAUACUCAC (SEQ |
| | | | ID NO: 31) |
| Full-length | GUGAGUAUACCGGU | GTGAGTATACCGGT | CUGAGGAAAAAAA |
| NEP- | GGAAGCGGUGCUAC | GGAAGCGGTGCTAC | AGGGAAAACCGGU |
| encoding | UAAUUUUUCACUUC | TAATTTTTCACTTCT | UUAUAUUAAUUGA |
| artificial | UCAAACAAGCAGGC | CAAACAAGCAGGC | AAGCUAAAAGUUC |
| intron | GAUGUGGAAGAAA | GATGTGGAAGAAA | UAAUUUCCUGUUC |
| | ACCCCGGACCAAUG | ACCCCGGACCAATG | CACUUCUAGAAGU |
| | GACCCAAAUACAGU | GACCCAAATACAGT | AAGUGUAGUGCUU |
| | CUCAUCAUUCCAAG | CTCATCATTCCAAG | GCAUGAAAGUGAU |
| | AUAUCCUGCUGAGG | ATATCCTGCTGAGG | CUGCUCAAAUGAA |
| | AUGUCCAAAAUGCA | ATGTCCAAAATGCA | UUCUCUGUAAUUU |
| | ACUCGAAUCGAGCA | ACTCGAATCGAGCA | UCAGUUUAUGUCG |
| | GUGGGGAUUUGAA | GTGGGGATTTGAAT | CACCUCUUCAAUA |
| | UGGCAUGAUUACAC | GGCATGATTACACA | AGCCAUCUUAUCU |
| | AAUUUGAAAGCCUG | ATTTGAAAGCCTGA | CUUCGAAUUUCUG |
| | AAGCUUUAUAGAG | AGCTTTATAGAGAC | GCCCAGUUGUUCG |
| | ACUCCCUUGGGGAA | TCCCTTGGGGAAGC | CGCCACUUCUCGU |
| | GCCGUGAUGAGAAU | CGTGATGAGAATGG | UCCUAUUCUGUAG |
| | GGGGGAUCUCCACU | GGGATCTCCACTCT | AGAGUGGAGAUCC |
| | CUCUACAGAAUAGG | CTACAGAATAGGAA | CCCAUUCUCAUCAC |
| | AACGAGAAGUGGCG | CGAGAAGTGGCGC | GGCUUCCCCAAGG |
| | CGAACAACUGGGCC | GAACAACTGGGCCA | GAGUCUCUAUAAA |
| | AGAAAUUCGAAGA | GAAATTCGAAGAG | GCUUCAGGCUUUC |
| | GAUAAGAUGGCUU | ATAAGATGGCTTAT | AAAUUGUGUAAUC |
| | AUUGAAGAGGUGC | TGAAGAGGTGCGAC | AUGCCAUUCAAAU |
| | GACAUAAACUGAAA | ATAAACTGAAAATT | CCCCACUGCUCGAU |
| | AUUACAGAGAAUUC | ACAGAGAATTCATT | UCGAGUUGCAUUU |
| | AUUUGAGCAGAUCA | TGAGCAGATCACTT | UGGACAUCCUCAG |
| | CUUUCAUGCAAGCA | TCATGCAAGCACTA | CAGGAUAUCUUGG |
| | CUACACUUACUUCU | CACTTACTTCTAGA | AAUGAUGAGACUG |
| | AGAAGUGGAACAG | AGTGGAACAGGAA | UAUUUGGGUCCAU |
| | GAAAUUAGAACUU | ATTAGAACTTTTAG | UGGUCCGGGGUUU |
| | UUAGCUUUCAAUUA | CTTTCAATTAATAT | UCUUCCACAUCGCC |
| | AUAUAAACCGGUUU | AAACCGGTTTTCCC | UGCUUGUUUGAGA |
| | UCCCUUUUUUUUCC | TTTTTTTTCCTCAG | AGUGAAAAAUUAG |
| | UCAG (SEQ ID NO: | (SEQ ID NO: 33) | UAGCACCGCUUCC |
| | 32) | | ACCGGUAUACUCA |
| | | | C (SEQ ID |
| | | | NO: 34) |

Importantly, the artificial introns of the present invention are designed such that, when the recombinant virus segments are transcribed into mRNA in a host cell, the artificial intron is spliced out of the mRNA at some frequency. To get spliced, the transcribed mRNA must include a “splice donor site” on the 5′ end of the intron, a branch site near the 3′ end of the intron, and “a splice acceptor site” on 3′ end of the intron. The splice donor site comprises an almost invariant GU sequence at the 5′ end of the intron, which exists within a larger, less highly conserved region. The splice acceptor site comprises an almost invariant AG sequence at the 3′ end of the intron. Upstream from the AG there is a region containing a high level of pyrimidines (C and U) or polypyrimidine tract. Upstream from the polypyrimidine tract there is a branchpoint, which includes the adenine nucleotide that is involved in lariat formation. However, the orientation of the intron is reversed within the viral segment sequence such that the intron comprises a donor site comprising the sequence AC on the 3′ end, a branch site near the 5′ end of the intron, and an acceptor site comprising the sequence CU on the 5′ end.

The sequences that can be used as 5′ and 3′ splice sites are variable but highly conserved. Thus, in the present application, these sequences are sometimes provided as consensus sequences that comprise symbols that represent variable nucleotides. The meaning of these symbols (i.e., which nucleotides each symbol represents) are detailed in Table 2.

TABLE 2
Variable nucleotide symbols

| Symbol | Represents |
|---|---|
| N | A, G, C, or T/U |
| R | G or A |
| Y | C or T/U |
| M | A or C |
| K | G or T/U |

For example, in some embodiments, the 3′ end of the artificial intron included in the recombinant viral segment comprises the consensus sequence ACUYAC, and in some embodiments, the 5′ end of the artificial intron comprises the consensus sequence CUGN followed by a 13-20 nucleotide long purine-rich region. In some embodiments, the ends of the artificial introns comprise one or more of the specific sequences that were used in the artificial introns tested in the Examples. Namely, the 3′ end of the artificial intron comprises the sequence ACUCAC and/or the 5′ end of the artificial intron comprises the sequence

(SEQ ID NO: 25)
CUGAGGAAAAAAAAGGGAAAA

Within the recombinant viral segments, the ends of the artificial intron form splice sites with the adjacent sequences of the viral segment. Thus, the surrounding viral sequences, which are referred to herein as the “splicing environment”, also play a role in splicing. In some embodiments, the artificial intron is inserted into the viral segment such that it is flanked on the 3′ end by the consensus sequence CUK and/or is flanked on the 5′ end by the consensus sequence NNC. In some embodiments, the splicing environment comprises one or more of the specific sequences that were used in the recombinant viral segments tested in the Examples. Namely, in some embodiments, the artificial intron is inserted into the genome segment such that it is flanked on the 3′ end by the sequence CUU and/or is flanked on the 5′ end by the sequence CAC.

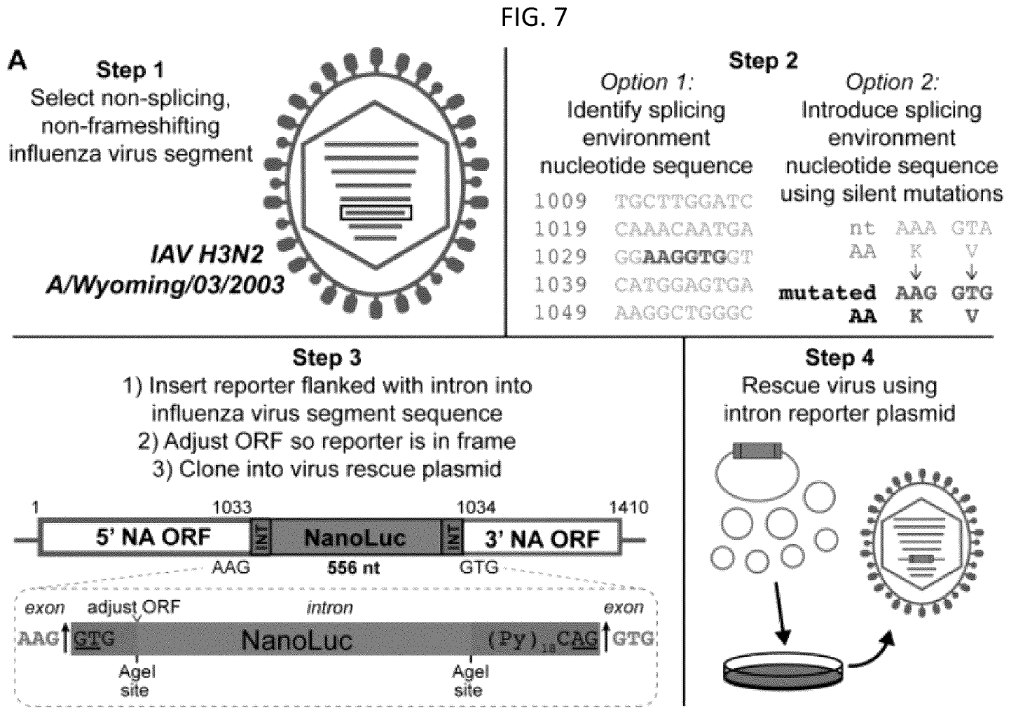

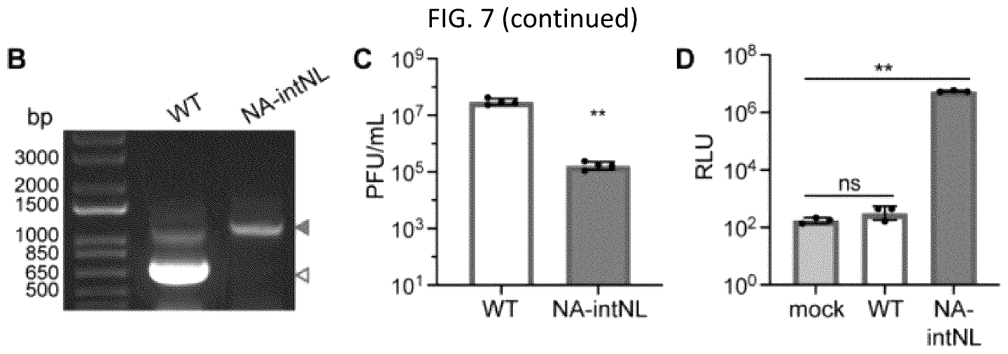

The process of generating a virus comprising an artificial intron is depicted in FIG. 7A. In some cases, appropriate splicing environment sequences are already present within a viral gene into which one wishes to introduce an artificial intron. In these cases, the artificial intron may simple be inserted into this splicing environment. However, in other cases, appropriate splicing environment sequences are not present within the viral gene. In these cases, these sequences may be introduced into the viral segment via silent mutations (i.e., mutations that change the nucleotide sequence but do not change the encoded amino acid). For example, in some embodiments, one or both of the CUK and NNC sequences used as the splicing environment were introduced into the viral segment via silent mutations. A list of silent mutations that can be used to produce the DNA sequence AAG GTG (i.e., the reverse complement of the viral segment sequence CAC CUU) is provided in Table 5.

As used herein, the term “branch site” refers to a sequence comprising the nucleotide that initiates a nucleophilic attack on the 5′ donor splice site during splicing. Branch sites are typically found 20-50 bases upstream of the 3′ end of an intron within an mRNA sequence. Thus, the branch site is found 20-50 bases downstream of the 5′ end of the intron within the recombinant viral segments described herein. In some embodiments, the branch site comprises the consensus sequence RUYAR. In some embodiments, the branch site comprises a specific sequence that was used in the artificial introns tested in the Examples. Namely, in some embodiments, the branch site comprises the sequence GUUAG.

Insertion of an artificial intron could be used to induce expression of alternative forms of the viral protein into which they are inserted. In Example 1, the inventors test an artificial intron (i.e., the constitutively spliced artificial intron) that encodes a stop codon, such that the spliced form of the protein is the full-length viral protein, whereas the unspliced form of the protein is truncated.

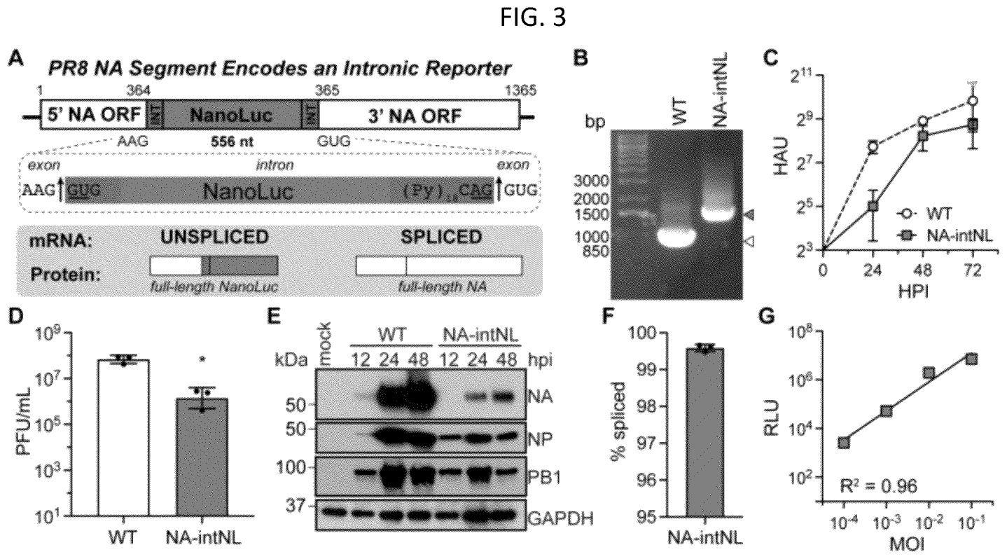

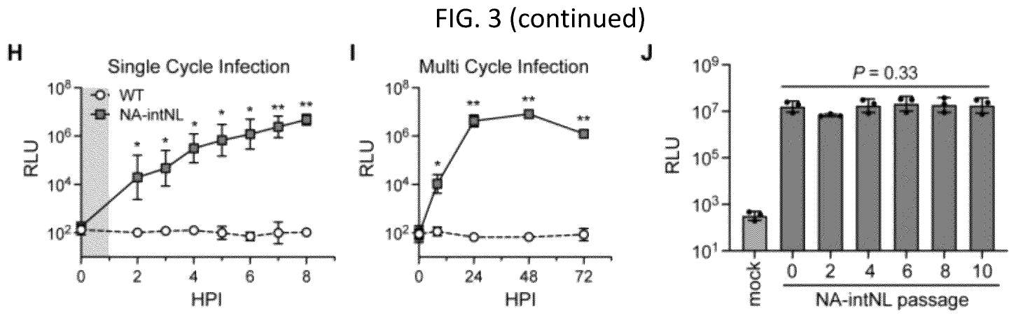

Alternatively, insertion of an artificial intron could be used to express a non-coding RNA (e.g., a short hairpin RNA or microRNA) or a protein of interest. In Example 1, the inventors demonstrate that a protein encoded by an artificial intron can be expressed from the recombinant viral segment following infection of a host cell (see FIG. 3). In fact, they demonstrate that both a heterologous protein encoded by an artificial intron and the protein encoded by the native viral gene into which the artificial intron was inserted are expressed in the host cell (see FIG. 3). Thus, the inventors have demonstrated that artificial introns can be used to expand the protein coding capacity of a virus.

Accordingly, in some embodiments, the artificial intron encodes a protein of interest. To ensure that this protein of interest is expressed, the artificial intron is inserted into an open reading frame (ORF) of a viral gene within the viral segment such that the portion of the artificial intron encoding the protein of interest is in the same reading frame as the ORF.

Any protein of interest may be encoded by the artificial intron for expression from a recombinant virus. Exemplary proteins of interest include, without limitation, reporter proteins and antigens.

A “reporter protein” is a protein that produces a trait or signal that is easily identified or measured. Exemplary reporter proteins are known in the art and include β-glucuronidase (GUS), an R-locus protein, a β-lactamase, a luciferase, a xy1E protein, an α-amylase, a tyrosinase, green fluorescence protein, and an α-galactosidase. In Example 1, the inventors demonstrate that the reporter protein NanoLuc® luciferase can be detectably expressed from an artificial intron (see FIG. 3). Thus, in some embodiments, the reporter protein is a luciferase.

As used herein, the term “antigen” refers to a molecule that can initiate a humoral and/or a cellular immune response in a recipient. Common categories of antigens include, but are not limited to, viral antigens, bacterial antigens, fungal antigens, protozoa and other parasitic antigens, tumor antigens, and antigens involved in autoimmune disease, allergy and graft rejection.

In some embodiments, the protein of interest is an antigen from a different segment of the virus. For example, if the recombinant viral segment comprises segment 6 of an influenza A virus, the protein of interest may be an antigen that is expressed from any other segment (i.e., segment 1-5, 7, or 8) of the influenza A genome. For instance, the inventors introduced an artificial intron encoding the influenza protein NEP/NS2 (which is natively expressed from segment 8) into segment 6 of the influenza A genome (see Example 2). Other suitable influenza A virus antigens include the proteins polymerase basic protein 2 (PB2), polymerase basic protein 1 (PB1), polymerase acidic protein (PA), hemagglutinin (HA), nucleoprotein (NP), neuraminidase (NA), matrix protein 1 (M1), matrix protein 2 (M2), non-structural protein 1 (NS1), and fragments thereof.

In other embodiments, the protein of interest is an antigen from a different virus. For example, if the recombinant viral segment is from the IAV strain A/Puerto Rico/8/1934, the protein of interest may be an antigen from another IAV strain (e.g., A/Wyoming/03/2003) or from an unrelated virus (e.g., SARS-CoV-2). Suitable viral antigens include proteins produced by viruses such as coronaviruses, alphaviruses, flaviviruses, adenoviruses, herpesviruses, poxviruses, parvoviruses, reoviruses, picornaviruses, togaviruses, orthomyxoviruses, rhabdoviruses, retroviruses, hepadnaviruses, herpesviruses, rhinoviruses, cytomegalovirus, Karposi sarcoma virus, human papillomavirus (HPV), human immunodeficiency virus (HIV), herpes simplex virus, herpesvirus 1, herpesvirus 2, herpesvirus 6, herpesvirus 7, herpesvirus 8, hepatitis A, hepatitis B, hepatitis C, measles, mumps, parvovirus, rabies virus, rubella virus, varicella zoster virus, Ebola virus, West Nile virus, yellow fever virus, dengue virus, rotavirus, Zika virus, and the like. For example, suitable viral antigens from severe acute respiratory syndrome coronavirus 2 (SARS-CoV-2) might include those derived from the spike (S), nucleocapsid (N), envelope (E), and membrane (M) structural proteins.

When an artificial intron encoding a protein of interest is inserted into an ORF of a viral gene within the viral segment such that it is in the same reading frame as the ORF, the unspliced transcription product produced from the recombinant viral segment will comprise the protein of interest fused to the 5′ sequence of the viral gene ORF. For Example, in the inventors' NA-intNL viruses (in which NanoLuc® luciferase is inserted into the NA gene in segment 6 of the IAV genome), the NanoLuc® reporter protein is fused with the stalk domain of the influenza protein NA (see FIG. 3A). In some cases, fusion of the protein of interest to the 5′ end of a viral protein may be advantageous because it may allow the protein of interest to be trafficked to the membrane and incorporated into the virion along with the 5′ end of the viral protein. For example, this arrangement may place the protein of interest in an ideal position to be recognized by the immune system for use as a vaccine. However, in some cases, this arrangement may be undesirable. For example, it may inhibit the function of the protein of interest or substantially reduce viral fitness. In these instances, a linker peptide may be incorporated into the artificial intron on the N-terminal end of the protein of interest. As used herein, the term “linker peptide” refers to a peptide sequence that bridges two protein components within a fusion protein. In some embodiments, the linker peptide is a “detachable linker”, i.e., a linker that results in the separation of the protein components flanking the linker. For instance, in Example 2, the inventors included a 2A cleavage-site on the N-terminal end of the protein of interest, which induces ribosomal skipping during translation of a protein in a cell, resulting in the production of two separate peptides. Suitable self-cleaving 2A polypeptides for use with the present invention include, without limitation, FMDV 2A, equine rhinitis A virus (ERAV) 2A (E2A), porcine teschovirus-1 2A (PTV1-2A), and Thoseaasigna virus 2A (T2A).

In some embodiments, a signal peptide is incorporated into the artificial intron on the N-terminal end of the protein of interest. As used herein, a secretion signal is a peptide motif that targets a protein to the secretory pathway of a cell. A signal peptide may cause a protein to which it is adjoined to be (1) targeted to a particular organelle (e.g., the endoplasmic reticulum, Golgi or endosomes), (2) secreted from the cell, or (3) inserted into the cellular membrane.

The viral segment from which the recombinant viral segment is derived may be from any negative-strand RNA virus from the Orthomyxoviridae or Bornaviridae family. A “negative-strand RNA virus” is a virus that has a genome comprising negative-sense, single-stranded RNA. The virus must be from the Orthomyxoviridae or Bornaviridae family because these viruses enter the nucleus of a host cell to replicate their genomes and transcribe viral mRNAs. This gives these viruses unique access to the host cell splicing machinery, which allows two distinct protein products to be produced from any viral segments comprising an intron. Suitable Orthomyxoviridae viruses for use with the present invention include, without limitation, influenza A, influenza B, influenza C, influenza D, isavirus, bourbon virus, salmon anemia virus, Thogotovirus, Dhori virus, and Quaranfil virus. Suitable Bornaviridae viruses for use with the present invention include, without limitation, Queensland carbovirus, Southwest carbovirus, Sharpbelly cultervirus, Elapid 1 orthobornavirus, Mammalian 1 orthobornavirus, Mammalian 2 orthobornavirus, Passeriform 1 orthobornavirus, Passeriform 2 orthobornavirus, Psittaciform 1 orthobornavirus, Psittaciform 2 orthobornavirus, and Waterbird 1 orthobornavirus. In the Examples, the inventors introduced artificial introns into various strains of influenza A. Thus, in some embodiments, the negative-strand RNA virus is an influenza A virus.

While it may be possible to introduce an artificial intron into any viral genome segment, for simplicity, the inventors selected viral segments that are not natively spliced and do not contain multiple overlapping reading frames (i.e., segment 5 and segment 6 of influenza A). Use of such segments ensures that the expression of viral proteins is minimally disrupted by the insertion of the artificial intron.

In some embodiments, the recombinant viral segment comprises one of the specific artificial intron sequences that was tested by the inventors. The tested artificial introns include a constitutively spliced artificial intron (SEQ ID NO: 28) that encodes a stop codon, an artificial intron that encodes the reporter protein NanoLuc® luciferase (SEQ ID NO: 31), and an artificial intron that encodes the influenza A protein NEP (SEQ ID NO: 34). The design of these introns was based on the artificial intron reported in a paper by Bonano et al. (Nature Protocols 2: 2166-2181, 2007), which is hereby incorporated by reference in its entirety. The present invention also encompasses artificial introns that are at least 99% identical, at least 98% identical, at least 95% identical, at least 90% identical, at least 85% identical, or at least 80% identical to SEQ ID NO: 28, 31, or 34.

### DNA Constructs:

In a second aspect, the present invention provides DNA constructs comprising the recombinant viral segments described herein.

As used herein, the term “DNA construct” refers to a recombinant DNA molecule, i.e., a DNA molecule that was formed artificially by combining at least two DNA components from different sources (natural or synthetic). For example, the DNA constructs of the present invention may comprise the coding region of one viral gene operably linked to an artificial intron encoding a protein that (1) is synthetic, (2) is from a different segment of the viral genome, or (3) is from a different organism. DNA constructs can be generated using conventional cloning methods.

In some embodiments, the DNA construct is a plasmid. A “plasmid” is a small, circular, double-stranded DNA molecule. Within a cell, a plasmid replicates independently from a cell's chromosomes.

As is described in greater detail in the section titled “Methods of making recombinant viruses” below, plasmid-based expression systems are commonly used to rescue infectious viruses. In such systems, a viral segment (in the form of cDNA) is inserted into a plasmid between an RNA polymerase I (pol I) promoter and a terminator sequence. This entire pol I transcription unit is flanked by an RNA polymerase II (pol II) promoter and a polyadenylation site. These plasmids comprising stacked pol I and pol II transcription units are referred to herein as “viral rescue plasmids”. The orientation of the two transcription units in the viral rescue plasmid allows for the synthesis of negative-sense viral RNA from one strand and positive-sense mRNA from the opposite strand, such that both viral RNAs and viral mRNAs/proteins are produced from the plasmid after it is transfected into a cell. Thus, in some embodiments, the DNA construct is a viral rescue plasmid.

### Recombinant Viruses:

In a third aspect, the present invention provides viruses comprising the recombinant viral segments described herein. The viruses are negative-strand RNA viruses from the Orthomyxoviridae or Bornaviridae family. Exemplary viruses from these virus families are listed in the section titled “Recombinant viral segments” above. In some embodiments, the virus was produced via rescue with a DNA construct described herein.

The recombinant viral segments described herein may be optimized for the codon usage of a specific virus. For example, influenza viruses have low GC content and preferentially utilize different codons than standard eukaryotes. Thus, to enhance expression and stability of any encoded proteins, the viral segments may be optimized using the publicly available Codon Optimization On-Line (COOL) or OPTIMIZER tool.

### Methods of Making Recombinant Viruses:

In a fourth aspect, the present invention provides methods of making a virus comprising an artificial intron. The methods comprise rescuing the virus with a DNA construct described herein.

“Virus rescue” is a technique that facilitates the generation of recombinant viruses. In this technique, each segment of the viral genome is cloned into a viral rescue plasmid in the form of cDNA. Specifically, the viral segment is cloned into a pol I transcription unit that is flanked by a pol II transcription unit in the viral rescue plasmid. Plasmids encoding each segment of the viral genome are transfected into a cell. In the cell, the plasmids are transcribed to produce negative-sense viral RNA from one strand and positive-sense mRNA from the opposite strand, such that all viral RNAs and mRNAs/proteins are expressed and packaged into viral particles (see, e.g., PNAS 99 (17) 11411-11416, 2002).

Thus, the methods of making a virus may comprise (a) introducing a DNA construct described herein into a viral rescue plasmid, (b) transfecting the resulting viral rescue plasmid into a cell along with viral rescue plasmids encoding each of the remaining viral segments needed to complete the viral genome, and (c) culturing the transfected cell to produce viral particles comprising the recombinant viral segment encoded by the DNA construct.

As used herein, the terms “transfecting” and “transfection” refer to a process of artificially introducing nucleic acids (DNA or RNA) into cells. Transfection may be performed under natural or artificial conditions. Suitable transfection methods include, without limitation, lipofection, bacteriophage or viral infection, electroporation, heat shock, microinjection, and particle bombardment.

The term “viral particle” refers to the extracellular phase of a virus. For example, an influenza viral particle consists of a nucleic acid core (i.e., the viral genome), an outer protein coating or capsid, and an outer envelope made of protein and phospholipid membrane derived from the host cell that produced the viral particle.

The cell lines that are transfected with the viral rescue plasmids in the present methods are eukaryotic cell lines. Suitable eukaryotic cells include, without limitation, mammalian cells or chicken cells. The cell may be a cell in culture or may be an embryonated chicken egg. Suitable mammalian cells include, without limitation, a MDCK cell, A549 cell, a CHO cell, a HEK293 cell, a HEK293T cell, a HeLa cell, a NS0 cell, a Sp2/0 cell, a COS cell, a BK cell, a NIH3T3 cell, a FRhL-2 cell, a MRC-5 cell, a WI-38 cell, a CEF cell, a CEK cell, a DF-1 cell, or a Vero cell.

The methods making a virus may further include additional steps that involve harvesting the influenza virus from the cell. In embodiments that utilize cultured cells, the methods may further comprise harvesting the supernatant of the culture by, for example, centrifugation or pipetting. In embodiments in which the cell is an embryonated chicken egg, the methods may further include harvesting the allantoic fluid from the embryonated chicken egg.

### Methods of Using Recombinant Viruses:

In a fifth aspect, the present invention provides methods for using the recombinant viruses described herein.

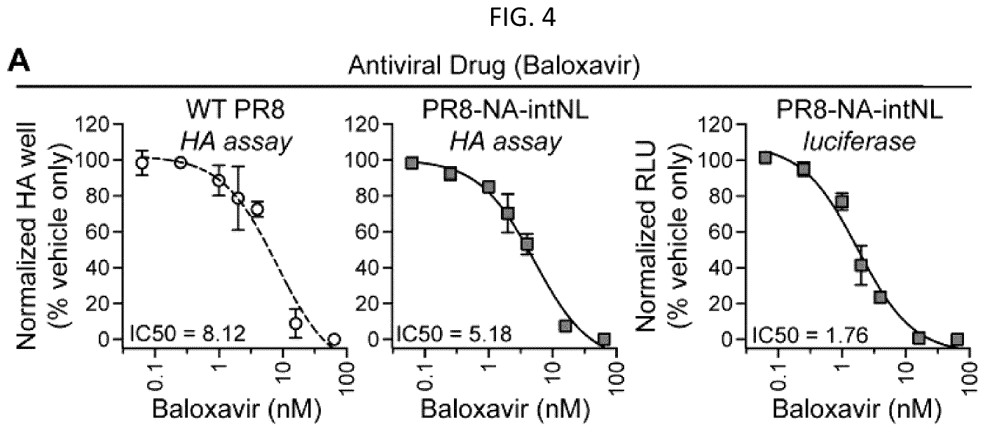

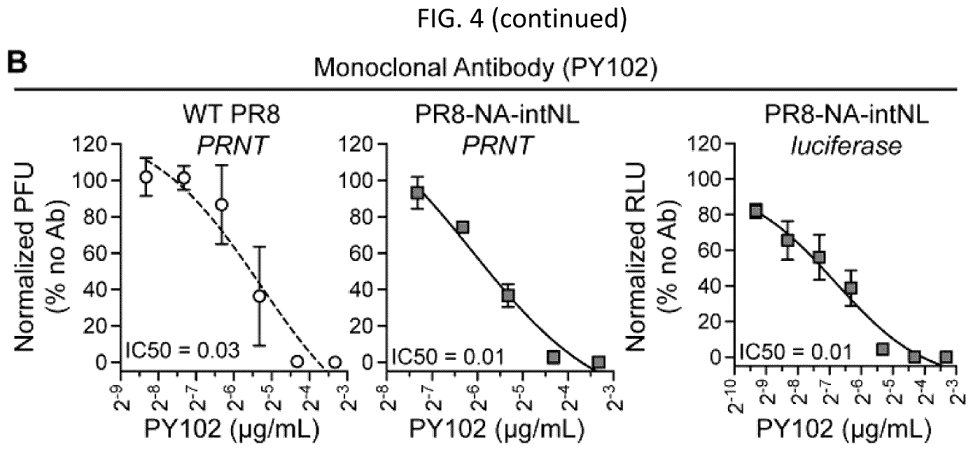

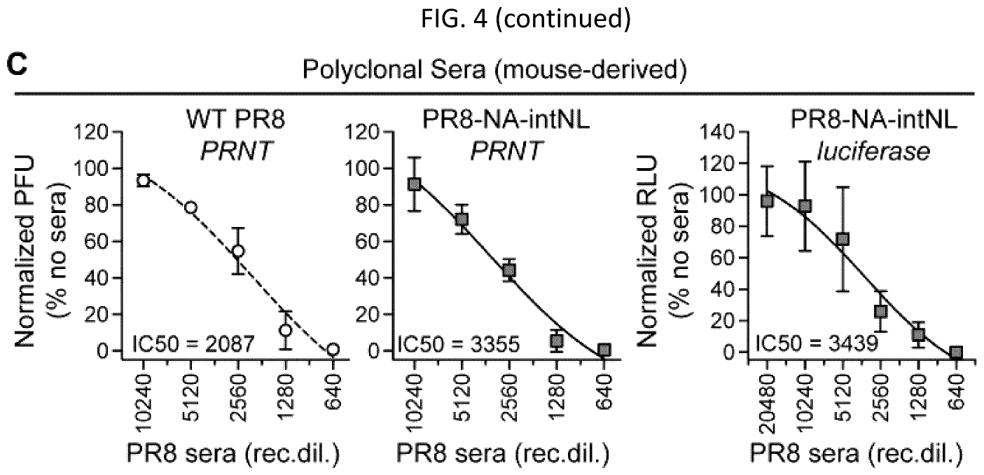

Embodiment 1: In one embodiment, the recombinant viruses are used in a screening assay. A “screening assay” is an assay that is used to identify compounds or reagents that have a desired biological activity. In Example 1, the inventors used a recombinant influenza A virus that expresses the reporter protein NanoLuc® luciferase from an artificial intron in a series of cell-based screening assays. Specifically, they measured viral inhibition by (1) the antiviral drug Baloxavir, (2) the neutralizing monoclonal antibody PY102, and (3) mouse derived anti-PR8 polyclonal serum using both hemagglutination assays and luciferase assays (see FIG. 4). Thus, in some embodiments, the screening assay is used to evaluate an antiviral drug, a neutralizing antibody (i.e., an antibody that binds to a virus in a manner that blocks infection), or immune sera (i.e., serum containing antibodies that is obtained from an animal that has been immunized with an antigen of interest).

Embodiment 2: In Example 2, the inventors introduced an artificial intron encoding the influenza protein NEP (which is natively expressed from segment 8) into segment 6 of the influenza A genome. Surprisingly, they found that insertion of this artificial intron enhanced the immunogenicity of the resulting recombinant virus, suggesting that such insertions may be used to create superior vaccines. Thus, in a second embodiment, the recombinant viruses are used to induce an immune response in a subject. These methods comprise administering a recombinant virus described herein to a subject.

In some embodiments, the recombinant virus is administered as part of a vaccine formulation.

The vaccine formulation may further comprise a pharmaceutically acceptable carrier. Pharmaceutically acceptable carriers are known in the art and include, but are not limited to, diluents (e.g., Tris-HCl, acetate, phosphate), preservatives (e.g., Thimerosal, benzyl alcohol, parabens), solubilizing agents (e.g., glycerol, polyethylene glycerol), emulsifiers, liposomes, and nanoparticles. Pharmaceutically acceptable carriers may be aqueous or non-aqueous solutions, suspensions, or emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions, or suspensions, including saline and buffered media.

The vaccine formulation may further include additives such as albumin or gelatin to prevent absorption to surfaces, detergents (e.g., Tween 20, Tween 80, Pluronic F68, bile acid salts), anti-oxidants (e.g., ascorbic acid, sodium metabisulfite), bulking substances or tonicity modifiers (e.g., lactose, mannitol). Components of the compositions may be covalently attached to polymers (e.g., polyethylene glycol), complexed with metal ions, or incorporated into or onto particulate preparations of polymeric compounds (e.g., polylactic acid, polyglycolic acid, hydrogels, etc.) or onto liposomes, microemulsions, micelles, milamellar or multilamellar vesicles, erythrocyte ghosts, or spheroplasts. The compositions may also be formulated in lipophilic depots (e.g., fatty acids, waxes, oils) for controlled or sustained release.

The vaccine formulation may also include adjuvants to increase their immunogenicity. Suitable adjuvants include, without limitation, mineral salt adjuvants, gel-based adjuvants, carbohydrate adjuvants, cytokines, or other immunostimulatory molecules. Exemplary mineral salt adjuvants include aluminum adjuvants, salts of calcium (e.g. calcium phosphate), iron, and zirconium. Exemplary gel-based adjuvants include aluminum gel-based adjuvants and acemannan. Exemplary carbohydrate adjuvants include inulin-derived adjuvants (e.g., gamma inulin, algammulin) and polysaccharides based on glucose and mannose (e.g., glucans, dextrans, lentinans, glucomannans, galactomannans). Exemplary cytokines include IFN-γ, granulocyte-macrophage colony stimulating factor (GM-CSF), IL-2, and IL-12. Suitable adjuvants also include any FDA-approved adjuvants for influenza vaccine usage including, without limitation, aluminum salt (alum) and the squalene oil-in-water emulsion systems MF59 (Wadman 2005 (Novartis)) and AS03 (GlaxoSmithKline).

In some embodiments, the vaccine formulation includes a concentration of total non-infectious viral particles of at least 10⁶ pfu/mL, at least 10⁷ pfu/mL, at least 10⁸ pfu/mL, at least 10⁹ pfu/mL, at least 10¹⁰ pfu/mL, or at least 10¹¹ pfu/mL.

In preferred embodiments, the methods comprise administering a therapeutically effective amount of the recombinant virus or vaccine formulation to the subject. As used herein, the term “therapeutically effective amount” refers to an amount of recombinant virus or vaccine formulation that is sufficient to induce an immune response in a subject receiving the recombinant virus or vaccine formulation.

An “immune response” is the reaction of the body to the presence of a foreign substance (i.e., an antigen). The immune response induced by the present methods may comprise a humoral immune response, a cell-mediated immune response, or both a humoral and cell-mediated immune response. The immune response of a subject to the recombinant virus may be evaluated through measurement of antibody titers or lymphocyte proliferation assays, or by monitoring signs and symptoms after challenge with the corresponding pathogen. The protective immunity conferred by the present methods may be evaluated by measuring a reduction in clinical signs, e.g., the mortality, morbidity, temperature, physical condition, or overall health of the subject.

In some embodiments, the immune response is against a native protein from the virus. For example, if the recombinant virus is derived from an influenza A virus, then the immune response may be against an influenza A protein. In some embodiments, the immune response is against a protein of interest encoded by the artificial intron. In these embodiments, the immune response may be against a heterologous protein that is not found in the virus in nature (e.g., an antigen from another virus or organism).

In some embodiments, the methods prevent or reduce the symptoms of influenza in the subject. The symptoms of influenza are well-known in the art and include, without limitation, headaches, chest discomfort, cough, sore throat, fever, aches, chills, fatigue, weakness, sneezing, and stuffy nose.

As used herein, the terms “administering” and “administration” refer to any method of providing a pharmaceutical preparation to a subject. Suitable routes of administration include, without limitation, intramuscular, intradermal, intranasal, oral, topical, parenteral, intravenous, subcutaneous, intrathecal, transcutaneous, nasopharyngeal, and transmucosal routes. In some embodiments, the recombinant virus is administered intramuscularly. The recombinant virus can be administered as a single dose or in multiple doses. For example, the recombinant virus may be administered two or more times separated by 4 hours, 6 hours, 8 hours, 12 hours, a day, two days, three days, four days, one week, two weeks, or by three or more weeks.

The “subject” to which the present methods are applied may any vertebrate. Suitable vertebrates include, but are not limited to, humans, cows, horses, sheep, pigs, goats, rabbits, dogs, cats, bats, mice, and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In preferred embodiments, the subject is a human.

Embodiment 3: In a third embodiment, the recombinant viruses are used to deliver a protein of interest or RNA of interest to a cell in a subject. These methods comprise administering a recombinant virus that comprises an artificial intron encoding the protein or RNA of interest to the subject.

In this embodiment, the protein of interest may be a therapeutic protein. As used herein, the term “therapeutic protein” refers to a protein (synthetic or naturally occurring) that induces a desired pharmacologic, immunogenic, and/or physiologic effect when administered to a subject. Exemplary therapeutic proteins include, without limitation, vaccine antigens, hormones, enzymes, cytokines, antibodies, receptors and antagonists, interferons, and the like.

An RNA of interest includes a short hairpin RNA, microRNA, antisense RNA, aptamer, ribozyme or other RNA molecule that may have a physiological effect in the cell, in the virus or in a subject after administration of the virus.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1

In the following example, the inventors describe the generation of influenza viruses comprising artificial introns. Influenza A viruses encode their genomes across eight, negative sense RNA segments. The six largest segments produce mRNA transcripts that do not generally splice. However, the two smallest segments are actively spliced to produce the essential viral proteins NEP and M2. Thus, viral utilization of RNA splicing effectively expands the viral coding capacity without increasing the number of genomic segments. As a first step towards understanding why splicing is not more broadly utilized across genomic segments, the inventors designed and inserted an artificial intron into the normally nonsplicing NA segment. This insertion was tolerated and, although viral mRNAs were incompletely spliced, they observed only minor effects on viral fitness. To take advantage of the unspliced viral RNAs, they next encoded a reporter luciferase gene in frame with the viral ORF such that when the intron was not removed the reporter protein would be produced. This approach, which they also show can be applied to the NP encoding segment and in different viral genetic backgrounds, led to high levels of reporter protein expression with minimal effects on the kinetics of viral replication or the ability to cause disease in experimentally infected animals. These data together show that the influenza viral genome is more tolerant of splicing than previously appreciated and this knowledge can be leveraged to develop viral genetic platforms with utility for biotechnology applications.

### Materials and Methods:

### Ethics Statement

Animal procedures were performed in compliance with IACUC approved protocols A189-18-08 and A142-21-07. Animals were assessed daily for signs of distress (change in respiratory rate, reduced movement, ruffled fur, change in grooming behaviors, agitation, lethargy) and bodyweight loss. Bodyweight loss of 20% compared to starting weight was the primary determinant of humane endpoints. CO₂ asphyxiation was used for primary euthanasia with bilateral thoracotomy as a secondary method.

### Cell Culture

Cells were obtained from ATCC and grown at 37° C. in 5% CO₂. Madin-Darby canine kidney (MDCK) cells were grown in minimal essential medium (MEM) with 5% fetal bovine serum (FBS), GlutaMax, HEPES, NaHCO₃, and penicillin-streptomycin. Human alveolar basal adenocarcinoma epithelial (A549) and chicken embryo fibroblasts (DF-1) cells were grown in Dulbecco's modified Eagle's medium (DMEM) with 10% FBS, GlutaMax, and penicillin-streptomycin. Human embryonic kidney 293T cells were grown in Dulbecco's modified Eagle's medium (DMEM) with 5% FBS, GlutaMax, and penicillin-streptomycin. All cells were maintained in plasmocin (2.5 μg/mL).

### Viruses

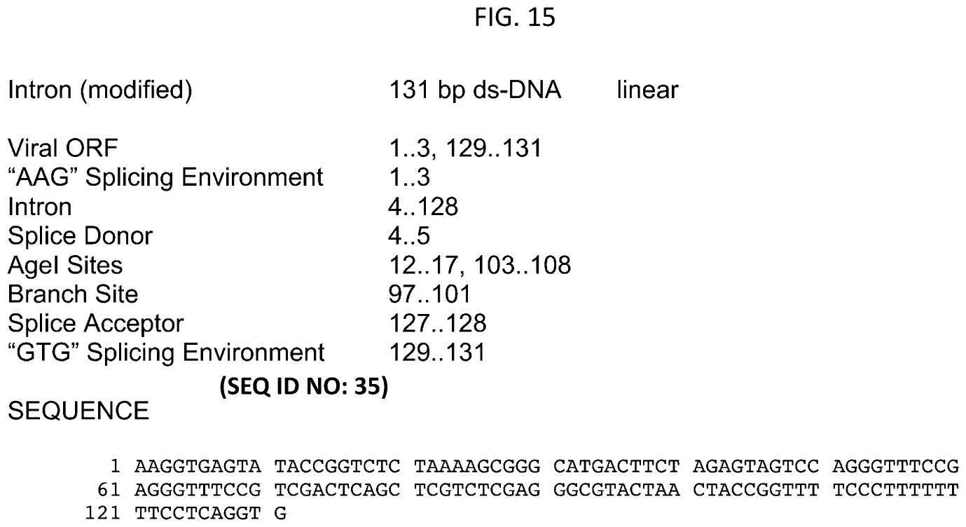

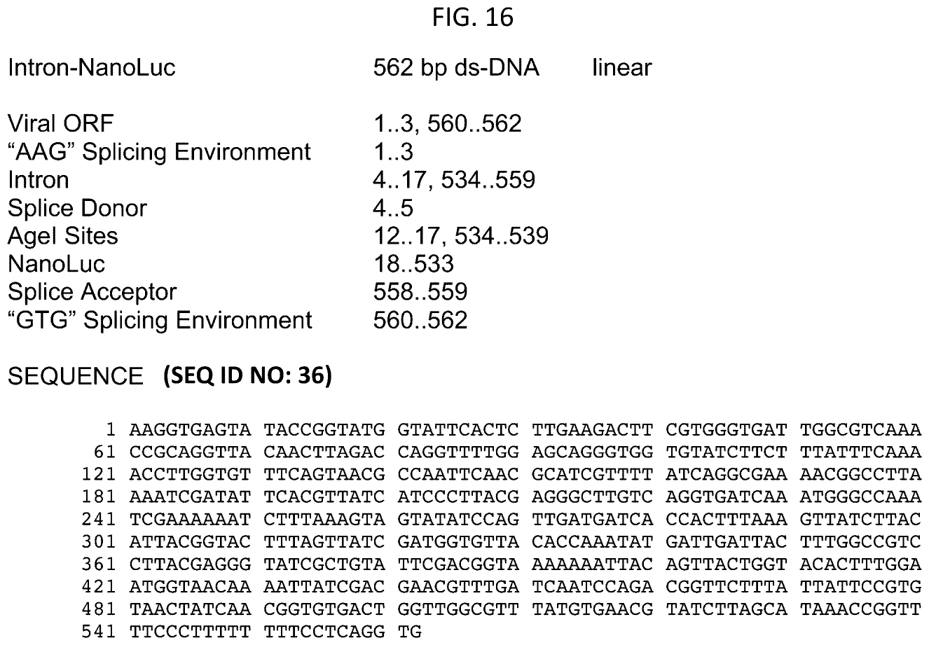

Recombinant viruses were generated by first inserting the desired segment with an intron sequence (FIG. 15 and FIG. 16) into the pDZ vector using platinum Taq PCR (Invitrogen) and the NEBuilder HiFi DNA assembly kit (New England BioLabs). A/swine/Henan/SN13/2018 HA (GenBank: MN416622) and NA (GenBank. MN416726) ORF sequences were cloned into PR8 packaging signals for their corresponding segment. DNA with the desired sequences were synthesized by Integrated DNA Technologies, Inc. The recombinant plasmids were transfected into low passage 293T cells with the WT versions of the remaining segments in pDZ plasmids using Trans-IT LT1 transfection reagent (Mirus). Supernatant was collected after 48 hours after transfection and used to infect MDCK cells. Supernatant from MDCK cells was collected 48-72 hours later and further used to infect MDCK cells or embryonated chicken eggs for plaque purification of rescued viruses. Unmodified A/Puerto Rico/8/1934 (PR8) and A/Wyoming/03/2003 (Wyo/03) viruses were grown and propagated in chicken eggs or on MDCK cells. Modified segments of viruses used in this study were reverse-transcribed and sequenced via Sanger sequencing.

### Cell Culture Infections

Cells were washed with phosphate-buffered saline (PBS) before being infected with virus diluted in PBS/BSA infection media. Cells were infected for 45 minutes and agitated every 10 minutes. Infection media was then removed and replaced with complete media for single cycle infections or post-infection media supplemented with TPCK trypsin for multicycle infections depending on experimental design.

### Hemagglutination (HA) Assays

Cell supernatant containing virus was diluted with cold PBS 1:2 for at least 8 dilutions in a V-bottom 96-well plate. 50 l of cold PBS containing a 1:40 dilution of chicken or turkey blood was added to the diluted virus wells, and the plate gently swirled to mix. Assays were incubated at 4° C. for at least 30 minutes before analysis. HA units were defined as the reciprocal of the highest dilution where hemagglutination was observed.

### Plaque Assays

MDCK cells were washed with PBS and then infected with 1:10 serially diluted virus for 45 minutes before virus was removed and replaced with an agar overlay. Cells were incubated at 37° C. for 48 hours before being fixed with 4% paraformaldehyde (PFA) in PBS for at least 3 hours. The agar overlay was then removed, and plaques were incubated overnight at 4° C. in sera or antibody diluted in antibody dilution buffer (5% nonfat dried milk, 0.05% Tween 20 in PBS). For viruses with PR8 glycoproteins anti-PR8 sera (derived from WT PR8 infected or immunized mice) was used; for 6+2 SW18 reassortant virus the anti-H1 stalk antibody 6F12 (mouse) was used; for Wyo/03 viruses the anti-H3 antibody 9H10 (mouse) in combination with anti-λ-31 sera (derived from X-31 infected or immunized mice) was used. Plaques were washed with PBS and then incubated for 1 hour in anti-mouse IgG horseradish peroxidase (HRP)-conjugated sheep (GE Healthcare) diluted in antibody dilution buffer. Plaques were washed with PBS and then stained with a TrueBlue peroxidase substrate (KPL) before being air-dried and counted.

### RT-PCR

Viral stocks, infection supernatants, infected egg allantoic fluid, or infected mouse lung homogenates were combined with Trizol (Ambion) and RNA was isolated and resuspended in nuclease-free water. Isolated RNA was reverse transcribed and amplified using SuperScript III One-Step RT-PCR System with Platinum Taq High Fidelity DNA Polymerase (Invitrogen) using primers targeting either the 5′ or 3′ region (900-1600 bp) of the segment of interest. RT-PCR samples were run on a 1% or 1.5% UltraPure agarose (Invitrogen) gel with SYBR Safe (Invitrogen) and imaged.

### Embryonated Chicken Egg Infections

10-day old chicken eggs were injected in the allantois with 100 μl virus diluted in PBS. The injection sites were sealed with wax and infected eggs were maintained at 37° C. until the designated collection time when eggs were moved to 4° C. overnight. Once eggs were completely cooled, the virus-containing allantoic fluid was collected.

### RNA Preparations for RT-qPCR

RNA samples from cell culture were prepared using the Monarch Total RNA Miniprep Kit (New England BioLabs). RNA samples from mouse lung homogenates were collected in Trizol (Ambion) and prepared according to the Phasemaker Tube protocol (Invitrogen).

### One-Step, Probe-Based RT-qPCR

RNA samples were analyzed using the EXPRESS Superscript One-Step qRT-PCR kit (Thermo Fisher) with primer/probes targeting the PR8 NA and NP RNAs (Table 3) (IDT) and eukaryotic 18S rRNA (Applied Biosystems) on an Applied Biosystems QuantStudio3 instrument.

TABLE 3
Primer and probe sequences for PR8
probe-based qPCR

| Name | Primer 1 | Primer 2 | Probe |
|---|---|---|---|
| PR8 | GAGTGACATCAAAA | CGTTCTCCATCAGTC | AGGCACCAAACGGTC |
| NP | TCATGGCG | TCCATC | TTACGAACA |
| | (SEQ ID NO: 1) | (SEQ ID NO: 2) | (SEQ ID NO: 3) |
| PR8 | GTTCCTGTTACCCT | TCACCGAAAACCCC | TGATCGAAGACACCC |
| NA | GATACCG | ACTG | ATGGCCG |
| | (SEQ ID NO: 4) | (SEQ ID NO: 5) | (SEQ ID NO: 6) |

### Two-Step, Dye-Based RT-qPCR

RNA samples were converted to cDNA with the PrimeScript RT reagent Kit (Perfect Real Time) (Takara) using only the included Oligo dT Primer. cDNA samples were analyzed using SsoAdvanced Universal SYBR Green Supermix (Bio-Rad) with unspliced/spliced isoform-specific primers targeting PR8 M and NS mRNAs and recombinant PR8 NA-intron/intNL and NP-intNL mRNAs (Table 4) on an Applied Biosystems QuantStudio3 instrument.

TABLE 4
Primer sequences for PR8 dye-based qPCR

| Name | Primer 1 | Primer 2 |
|---|---|---|
| M1/2 | TGCATTTACCGTCGCTTTA | CCTCATAGACTTTGGCACTC |
| | (SEQ ID NO: 7) | (SEQ ID NO: 8) |
| M1 | GCACTACAGCTAAGGCTATG | GAGTCCCAATGGTTCTCATC |
| only | (SEQ ID NO: 9) | (SEQ ID NO: 10) |
| NS1/ | CATACTGCTGAGGATGTCAAA | CAGAGACTCGAACTGTGTTATC |
| NEP | (SEQ ID NO: 11) | (SEQ ID NO: 12) |
| NS1 | CTTCGCCGAGATCAGAAATC | TCAGAATCCGCTCCACTATC |
| only | (SEQ ID NO: 13) | (SEQ ID NO: 14) |
| NA- | GGAGCAGTGGCTGTATTAAA | CGGGCCATCAGTCATTATAG |
| int/NA | (SEQ ID NO: 15) | (SEQ ID NO: 16) |
| NP- | GCCAGAATGCCACTGAAA | GATCAACCGTCCCTCATAATC |
| int/NP | (SEQ ID NO: 17) | (SEQ ID NO: 18) |
| NA-int | TCCCTTTTTTTTCCTCAGGTG | ATTAAGGCCCTATAAGGGCT |
| only | (SEQ ID NO: 19) | (SEQ ID NO: 20) |
| NP-int | TCCCTTTTTTTTCCTCAGGTG | AGTACCTGCTTCTCAGTTCA |
| only | (SEQ ID NO: 21) | (SEQ ID NO: 22) |

### Western Blotting

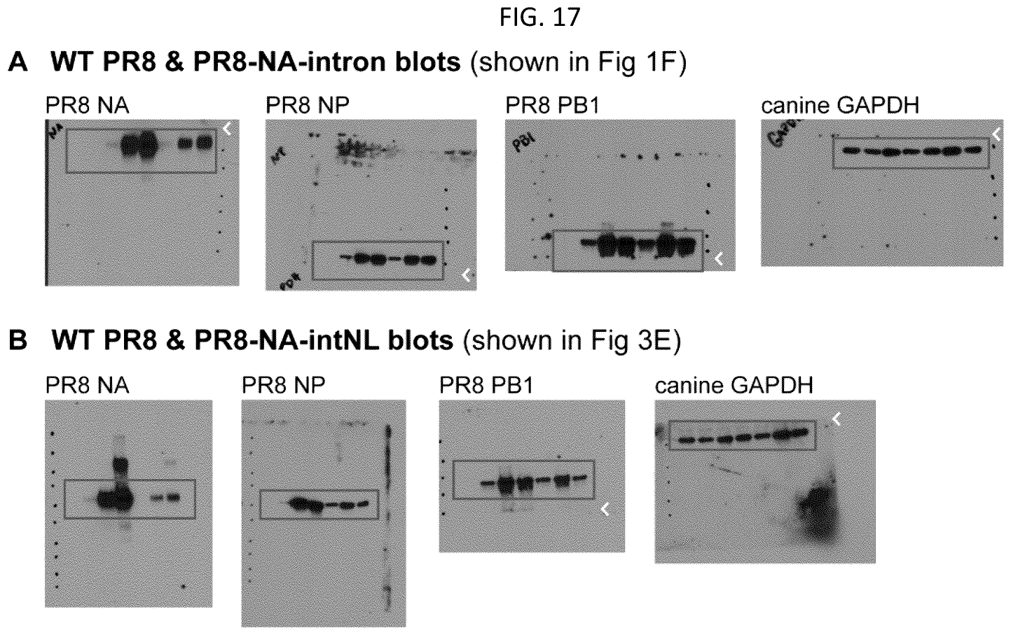

Protein samples were collected via chemical cell lysis using RIPA buffer (10 mM Tris-HCl pH 7.5, 1 mM EDTA pH 8.0, 1% Triton X-100, 0.1% sodium deoxycholate, 140 mM NaCl, 0.1% SDS) and normalized by total protein concentration before adding SDS-PAGE sample buffer (Bio-Rad). Protein samples were loaded and run on a 4-20% polyacrylamide gels (Bio-Rad). Gels were transferred to nitrocellulose membranes before being blocked with PBS containing 5% (w/v) non-fat dried milk and 0.1% Tween-20 for at least 1 hour at room temperature or overnight at 4° C. Membranes were incubated with primary antibody diluted in PBS containing 5% (w/v) non-fat dried milk and 0.1% Tween-20 for at least 1 hour at room temperature or overnight at 4° C. overnight. Primary antibodies used included anti-N1 (4A5), anti-NP (GeneTex GTX125989), anti-PB1 (GeneTex GTX125923), and anti-GAPDH (Abcam ab181603). Membranes were washed 3 times with PBS containing 0.1% Tween-20 before being incubated with anti-mouse-HRP (Invitrogen A16072) or anti-rabbit-H1RP (Invitrogen A16104) secondary antibodies for 1 hour at room temperature. Membranes were washed 3 times with PBS containing 0.1% Tween-20 before treatment with Clarity or Clarity Max ECL (Bio-Rad) and exposure to film for development. Uncropped Western blots are shown in FIG. 17.

### Luciferase Assays

Infected cells were lysed in 1× Luciferase Cell Lysis Reagent (Promega) while shaking at room temperature for 20 minutes and then moved to a 96-well V-bottom plate. Settled samples were moved to luminometer tubes. Nano-Glo Luciferase Assay Kit (Promega) reagents were prepared and combined with lysed cells, egg allantoic fluid, or mouse lung homogenates for a standard amount of time before being read using an EG&G Berthold Lumat LB 9507 machine. If samples read overload, all samples in that experiment were diluted 1:10 and reread and their reported values were multiplied by 10 to reflect the dilution factor.

### Plaque Reduction Neutralization Tests (PRNTs)

Viruses were incubated with PY102 antibody (mouse anti-PR8 HA) or anti-PR8 sera (derived from WT PR8 infected or immunized mice) dilutions in PBS/BSA for 45 minutes. MDCK cells were washed with PBS then infected with the virus/antibody or virus/sera dilutions for 45 minutes before virus was removed and replaced with an agar overlay supplemented with TPCK trypsin. The infected plates then incubated for 48 hours at 37° C. before being fixed with 4% paraformaldehyde (PFA) in PBS for at least 3 hours. The agar overlay was then removed, and plaques were incubated overnight at 4° C. with anti-PR8 sera (derived from WT PR8 infected or immunized mice) diluted in antibody dilution buffer (5% nonfat dried milk, 0.05% Tween 20 in PBS). Plaques were washed with PBS and then incubated for 1 hour in anti-mouse IgG horseradish peroxidase (HRP)-conjugated sheep (GE Healthcare) diluted in antibody dilution buffer. Plaques were washed with PBS and then stained with a TrueBlue peroxidase substrate (KPL) before being airdried and counted.

### Animal Infections

6- to 12-week-old age matched BL/6 female mice from Jackson Laboratories were anaesthetized using an injection of ketamine/xylazine. Tails were marked and mice were weighed before being intranasally infected with 40 l virus diluted in pharmaceutical grade PBS. Mice were weighed daily and euthanized if their body weight reached less than 80% of their starting weight. All procedures were completed according to IACUC.

### Data Analysis and Presentation

For all experiments, the statistical analyses used to compare experimental groups are indicated in the corresponding figure legends and were performed using GraphPad Prism. All graphs include data from (and statistical analyses were performed on) 3 independents experiments or ≥3 independent biological entities for egg- and mouse-derived data. Western blots and RT-PCR gel images (of passaged virus experiments) shown are representative of three independent experiments. In cases where values were undetermined or below the limit of detection, statistical analyses were performed using only the detected values-if no values were detected for a given datapoint, it is indicated as not detected (ND) within the graph. In some cases where a viral time course is shown, no pre-infection (0 h) experimental samples were collected and the line connecting datapoints simply starts at the graph origin. Data displayed on a log 10 scale was log transformed, plotted, and analyzed as linear data, and graphed on a power of 10 axis. Data displayed on a log 2 scale was plotted, analyzed, and graphed on a log 2 axis.

### Results:

### StIAV Segment 6 Tolerates Introduction of an Artificial Intron

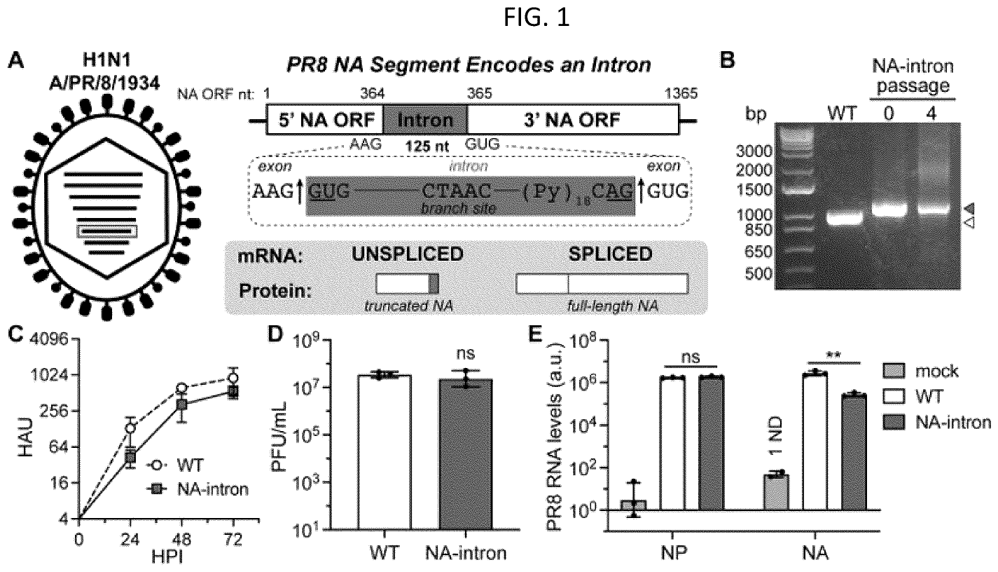

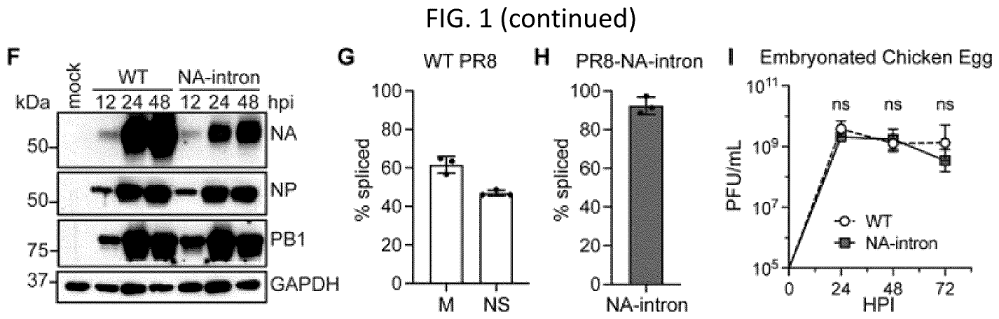

To investigate whether normally nonsplicing viral RNAs can tolerate splicing during a viral infection, we aimed to introduce an artificial intron via reverse genetics. To accomplish this goal we selected a constitutively spliced intron sequence [22] with the idea that, after insertion into the viral segment, the dominant mRNA species would encode a functional viral protein, rather than the intron-retained, nonfunctional version. We selected the H1N1 A/Puerto Rico/8/1934 (PR8) strain segment 6 (which encodes the viral neuraminidase, NA) as the intron target because it is the next shortest segment after the spliced segments 7 and 8 and the increased genomic segment length would not exceed the length of the longest viral segments. To generate the intron-containing segment, we identified a six-nucleotide sequence, “AAGGUG,” within the NA coding region. We inserted the constitutively spliced intron sequence after the PR8 NA encoded “AAG,” forming part of a splice donor site, and before the encoded “GUG,” forming part of a splice acceptor site (FIG. 1A). As designed, the spliced version of this PR8 NA-intron mRNA should be identical to the wild-type (WT) PR8 NA mRNA. The unspliced version of the PR8 NA-intron mRNA retains the intron and encodes a stop codon, resulting in a truncated protein product (FIG. 1A). We rescued this virus in the PR8 background (PR8-NA-intron) and determined the stability of our PR8-NA-intron virus over four serial passages on MDCK cells and observed no loss in segment length (FIG. 1B). We also found it grew to high titers without a significant growth defect compared to WT PR8 under multicycle growth conditions on MDCK cells (FIGS. 1C and 1D). Together, these findings demonstrate that IAV PR8 tolerates the introduction of a highly spliced intron in segment 6.

The presence or absence of introns in influenza virus mRNAs is recognized to impact their transport and translation [4]. To determine if the addition of an intron impacted the transcription, replication, or translation of NA, we measured the RNA (using an assay that would not discriminate between mRNA, vRNA, and cRNA) and protein levels from WT PR8 or PR8-NA-intron virus-infected MDCK cells. We found that there was a modest reduction in NA RNA expression levels and a corresponding decrease in NA protein levels between our PR8-NA-intron virus and the WT PR8 virus (FIG. 1E-F).

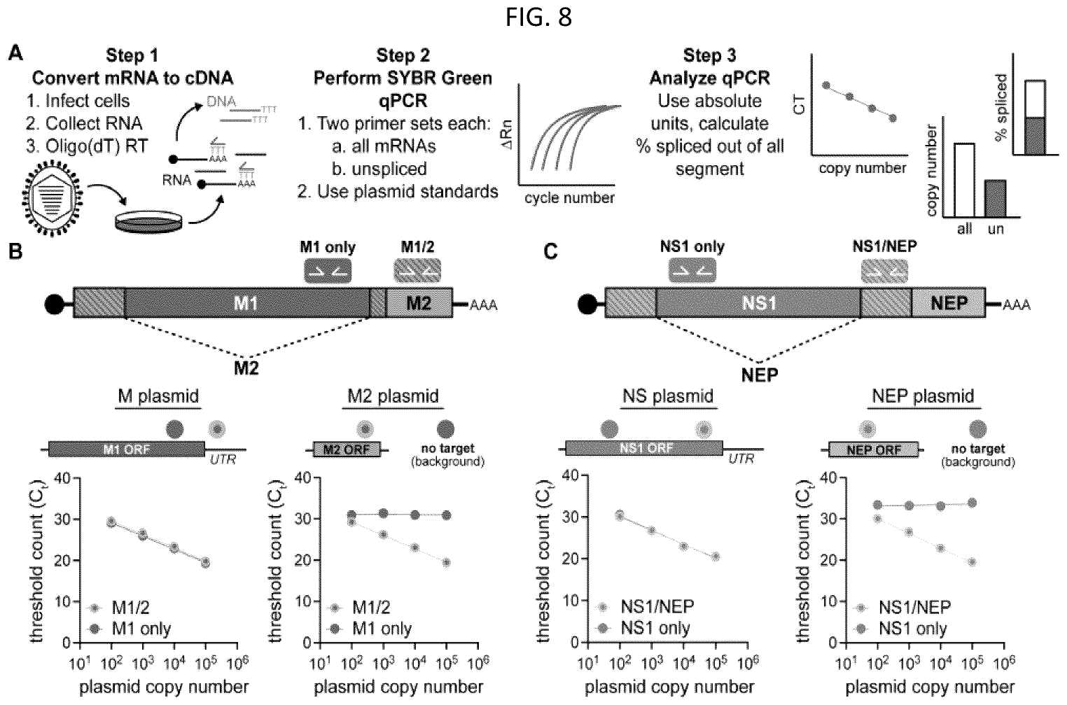

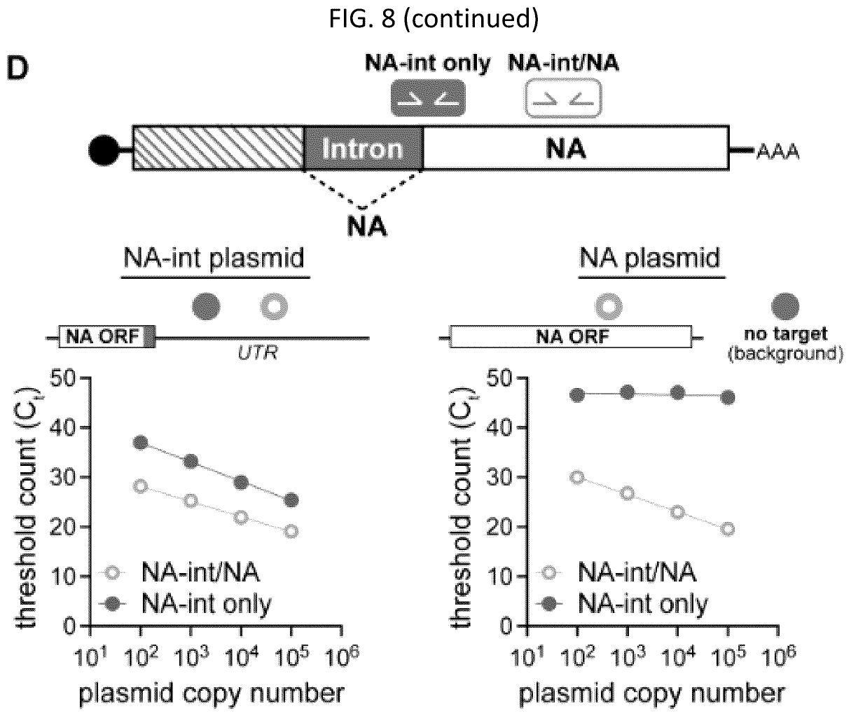

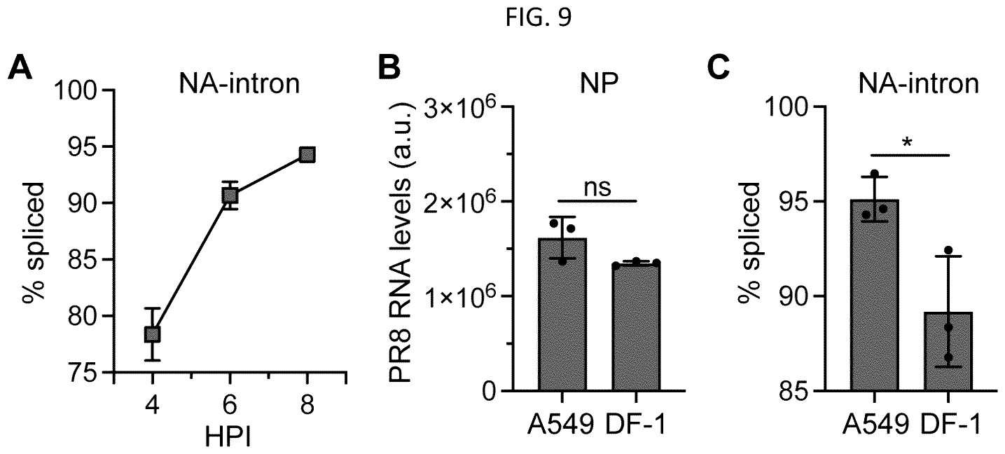

We designed the PR8 NA-intron segment to be highly spliced while endogenous influenza intronic sequences are often retained to reflect the protein needs of a replicating virus [5]. Therefore, we expected our introduced segment 6 intron to be spliced at a higher rate than the endogenous introns in IAV segments 7 and 8. We observed splicing rates around 60% and 40% for the WT PR8 M and NS segments, respectively, during a WT PR8 virus infection in MDCK cells (FIG. 1G and FIG. 8A-C). In contrast, our constitutively spliced artificial intron was spliced in about 90% of NA-intron mRNA transcripts (FIG. 1H and FIG. 8D). Time also dictates IAV mRNA splicing rates and protein balance during infection; early in infection, M1 is expressed more highly, while M2 levels increase later in infection, indicating that splicing increases as infection progresses [23]. We were interested in whether this time-dependent increase in the splicing of influenza viral mRNAs would apply to our newly introduced intron in the NA segment. Indeed, we observed increased splicing of the PR8 NA-intron mRNAs over time (FIG. 9A).

Splicing machinery is generally conserved among vertebrate species; however, splicing is also a recognized host determinant for avian- and mammalian-derived influenza viruses [9-13]. Most notably, avian-adapted influenza viruses have been reported to replicate poorly in mammalian cells due to excessive M splicing [24,25]. Therefore, we were interested in how our NA-intron, which was not specifically adapted to either an avian or mammalian host, would behave in different hosts. We first infected embryonated chicken eggs with WT PR8 and PR8-NA-intron virus and found no observable defect in infectious viral production, suggesting successful viral replication in an avian environment (FIG. 1I). To more rigorously define the potential differences between mammalian and avian growth, we infected human lung epithelial cells (A549) and chicken embryo fibroblast cells (DF-1) with the PR8-NA-intron virus. The replication of PR8-NA-intron was similar in both cell types (FIG. 9B). However, previous findings show that avian IAV M mRNAs are more frequently spliced in mammalian cells compared to avian cells [24,25] and we similarly observed significantly more splicing of the NA-intron segment in the human A549 cells compared to avian DF-1 cells (FIG. 9C). These findings indicate that many of the same factors which dictate the canonical splicing of IAV M mRNAs over time and in different hosts likely govern splicing of our artificial intron.

### Artificial Introns of Different Lengths are Tolerated and can be Engineered to Express Exogenous Proteins

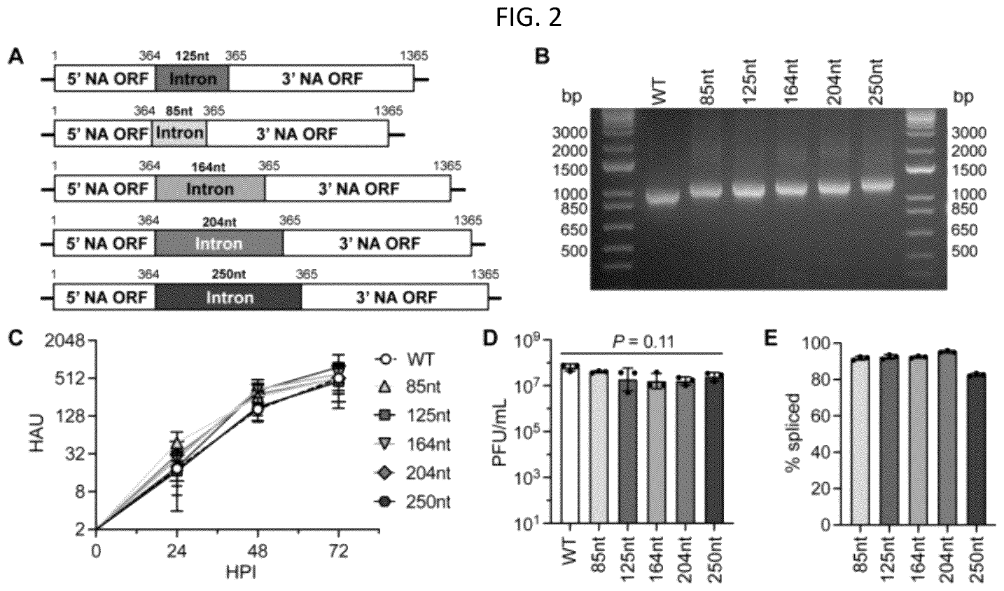

Intron length and cis-elements, both intronic and exonic, are important splicing determinants [26,27]. We therefore wanted to test if the ability of a viral segment to tolerate segment splicing was dependent on the specific characteristics of the intron. As a way to modify the intron itself we first varied the length of the intron, originally 125 nt, to 85 nt, 164 nt, 204 nt, or 250 nt, in the NA segment and rescued the corresponding viruses in the PR8 background (FIG. 2A-B). Each PR8-NA-intron virus, regardless of intron length, grew to high titers after multicycle infection on MDCK cells (FIG. 2C-D). Additionally, the intron-retained, unspliced mRNA remained the minor product compared to the spliced, functional viral protein encoding mRNA (FIG. 2E).

Since our artificial introns were spliced in ˜90% of mRNAs (FIG. 1F), we next wanted to determine if we could use the remaining ˜10% of transcripts retaining the intron to expand the coding capacity of the IAV genome. Therefore, we selected the NanoLuc gene as a model ORF for insertion based on its small size (516 nt) and detectable activity at low levels of expression [28,29]. We inserted a constitutively spliced, intron-flanked NanoLuc gene in frame at the previous location in the PR8 NA segment such that the unspliced mRNAs would now express the NanoLuc protein (FIG. 3A). We successfully rescued the virus in the PR8 genetic background and showed that, while the PR8-NA-intNL virus had delayed growth kinetics relative to WT virus, it grew to high, but somewhat reduced, titers (FIG. 3B-D). As expected, the reduced kinetics of replication and lower end point titers could also be observed at the viral protein level over time (FIG. 3E). Unexpectedly, splicing of the NA mRNA increased with the larger, reporter-containing intron, from an ˜90% splicing rate in our PR8-NA-intron virus to more than 99% spliced in the PR8-NA-intNL virus (FIG. 3F). Despite the low production of NanoLuc-containing transcripts, luciferase activity correlated well with infectious dose and high levels of reporter gene expression were detectable during single- and multi-cycle virus infections on MDCK cells (FIG. 3G-I). Thus, genes can functionally be expressed from artificial intron-containing influenza viral segments.

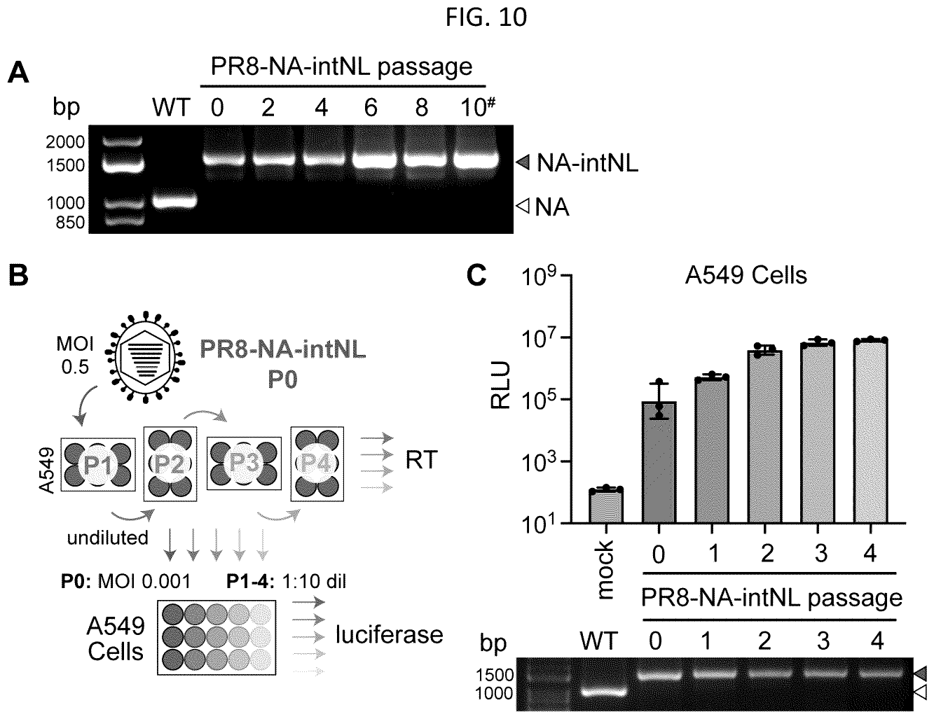

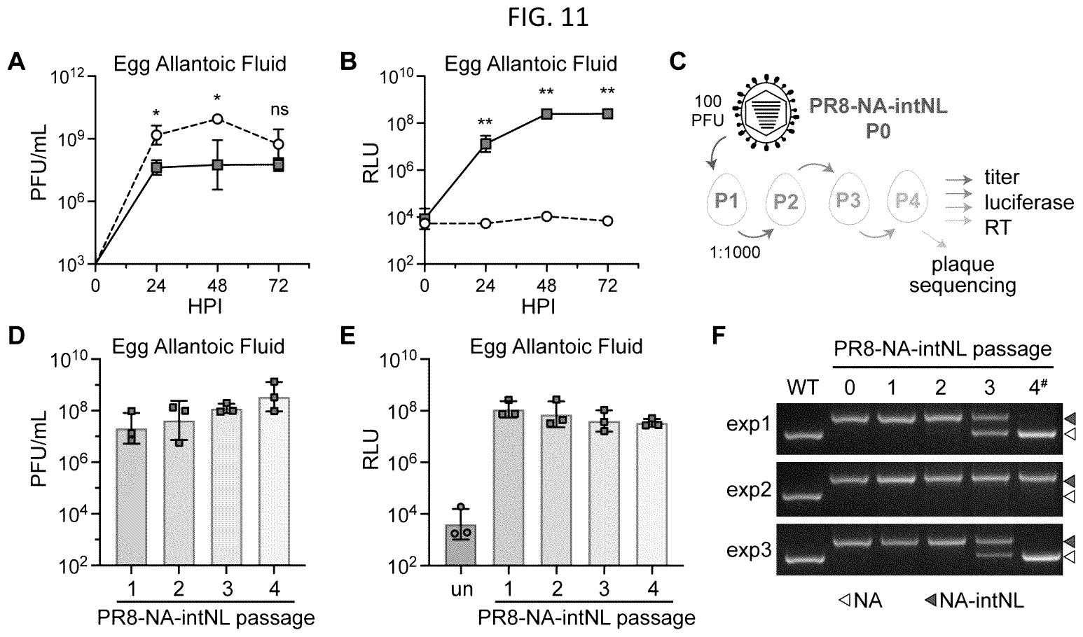

For intronic reporter viruses to have practical utility they must be stable throughout an experiment and ideally through multiple rounds of propagation. We therefore expanded our passaging experiments and found that, after 10 passages of the PR8-NA-intNL virus on MDCK cells, luciferase activity remained insignificantly changed from the virus stock and RT-PCR and sequencing of PR8 segment 6 demonstrated that the intronic NanoLuc reporter was stable (FIG. 3J and FIG. 10A). After passaging on human lung A549 cells we observed similar results to the MDCK cell experiments (FIG. 10B-C). We next tested viral growth and stability in embryonated chicken eggs. While the PR8-NA-intNL virus replicated and produced high levels of luciferase in this environment, the intron was sometimes lost after multiple rounds of serial passage (FIG. 11A-F). Thus, mammalian culture methods are preferable for propagating intronic reporter viruses.

### Intron-Reporter Containing Viruses can be Used for Cell-Based Screening Assays

Luciferase reporter viruses have previously been utilized in many applications, including as tools for influenza virus antiviral drug, neutralizing antibody, and immune sera screening [30-35]. We next tested our reporter virus in these contexts relative to unmodified, wild-type virus. First, we measured the effect of a recognized influenza antiviral Baloxavir, a cap-dependent endonuclease inhibitor that blocks influenza PA activity [36]. Using a hemagglutination assay readout, we found both viruses were inhibited at similar drug levels (FIG. 4A). We also collected PR8-NA-intNL virus-infected, Baloxavir-treated cells for luciferase assays and, using luciferase signal, determined a comparable inhibitory concentration (FIG. 4A). We then performed plaque reduction neutralization tests (PRNTs) using the anti-PR8 neutralizing monoclonal antibody PY102 [37] with WT PR8 and PR8-NA-intNL viruses and found the neutralizing antibody inhibited both viruses with similar IC50s (FIG. 4B). Decreased reporter activity from MDCK cells infected with PR8-NA-intNL virus that were pre-incubated with PY102 antibody also correlated well with the antibody-based inhibition of infectious virus levels (FIG. 4B). Analogous experiments using mouse-derived, anti-PR8 polyclonal serum showed our intron-reporter virus is also suitable for neutralizing sera-based experiments (FIG. 4C). Thus, intronic reporter viruses have utility in many common reporter virus assays.

### An Intron-Reporter IAV Strain Replicates and can Cause Disease In Vivo

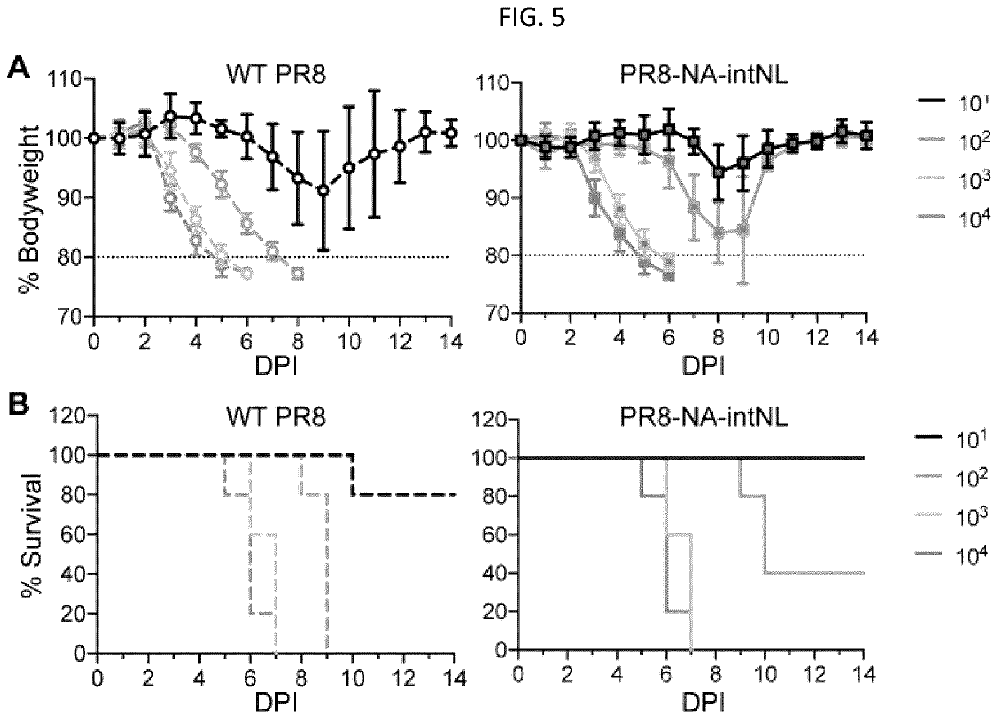

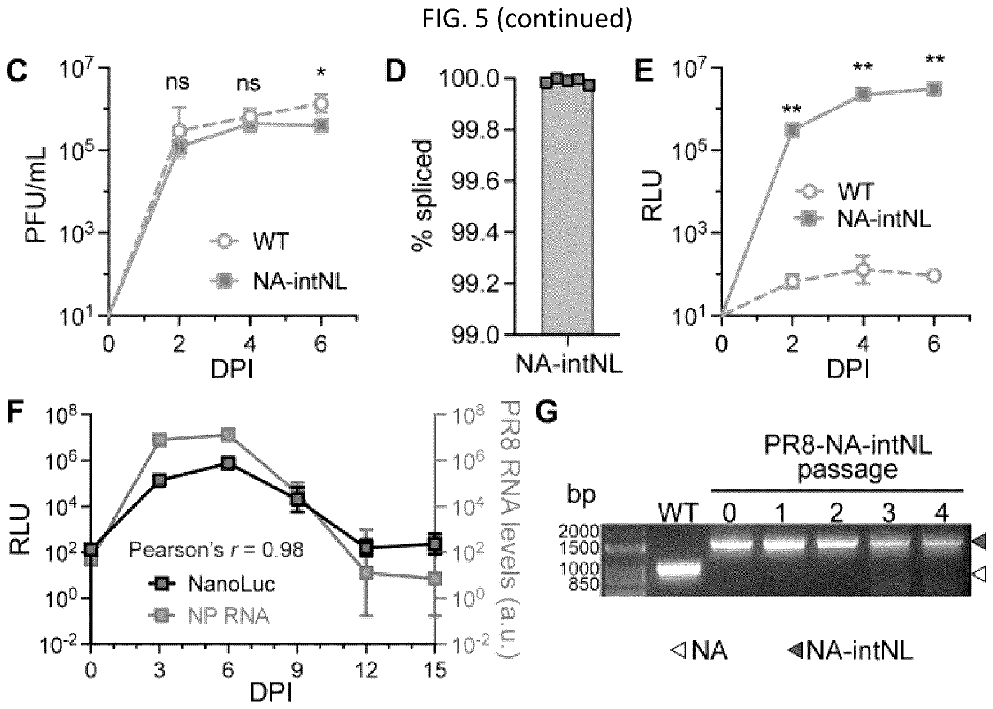

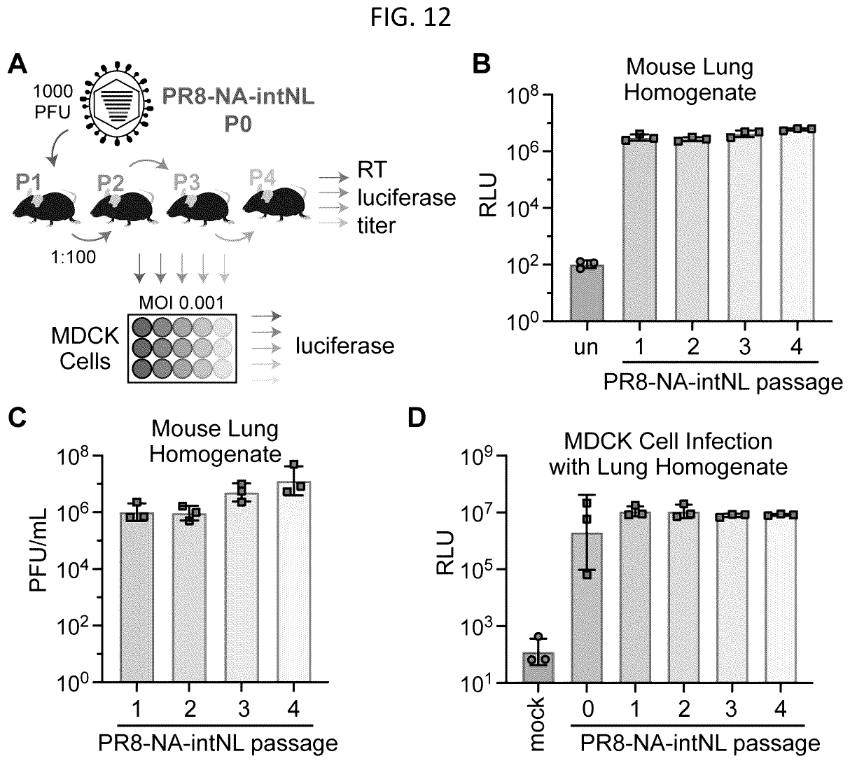

Cancer cells are known to alter the cellular splicing environment [38], and most of our previous experiments had been performed in immortalized cancer cell lines. As a result, we were interested in how the inclusion of an intron in an additional viral segment would impact in vivo influenza virus infections. We therefore infected immune competent C57BL/6 mice with a range of doses of WT PR8 virus or the PR8-NA-intNL virus and measured their bodyweight loss as an indicator of disease. The PR8-NA-intNL virus resulted in both mouse weight loss and mortality, though at higher viral doses compared to WT PR8 (FIG. 5A-B). Lung virus growth kinetics were similar between WT PR8 and PR8-NA-intNL virus infections at a potentially lethal viral dose (100 PFU) (FIG. 5C). Despite a high degree of intron-reporter splicing (greater than 99% spliced) (FIG. 5D), we detected luciferase activity from mouse lung homogenates following the pattern expected from viral growth kinetics (FIG. 5E). We also infected mice with a sublethal dose of the PR8-NA-intNL virus (10 PFU) and observed a strong correlation (r=0.98) between luciferase activity and viral RNA at all timepoints (FIG. 5F). Finally, we serially passaged the PR8-NA-intNL virus in mouse lungs and found the NanoLuc reporter remained stable (FIG. 5G and FIG. 12A-D).

### An Intron-Encoded Reporter Gene is Also Tolerated in the IAV NP Segment

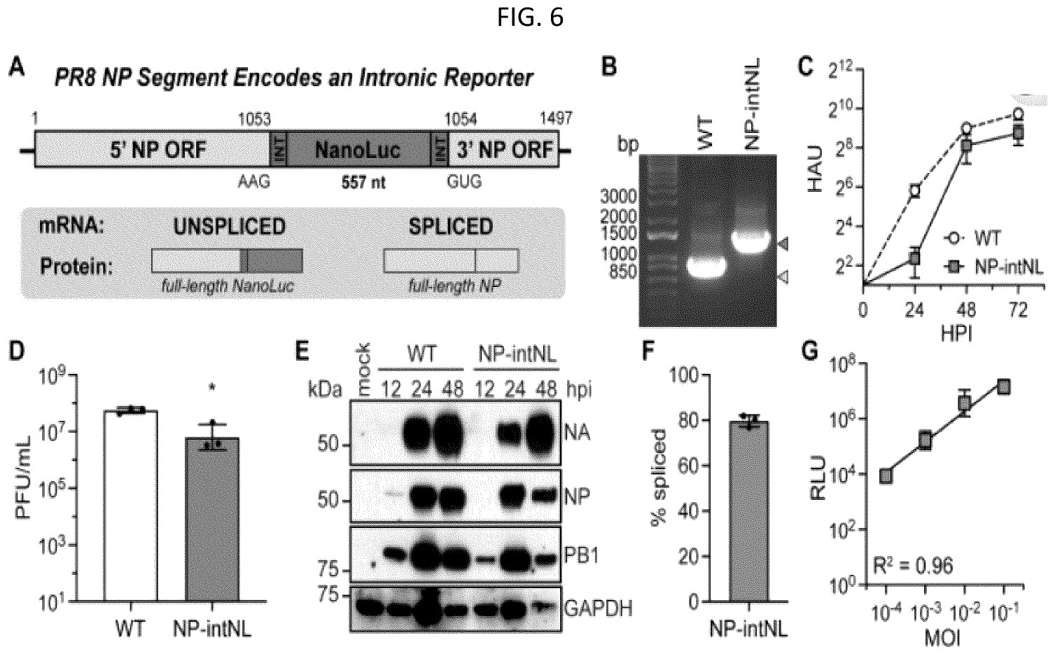

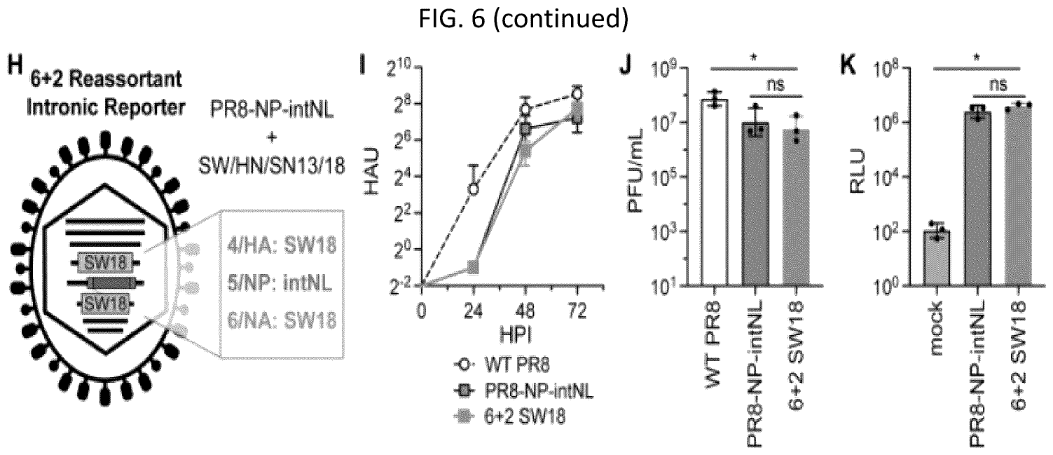

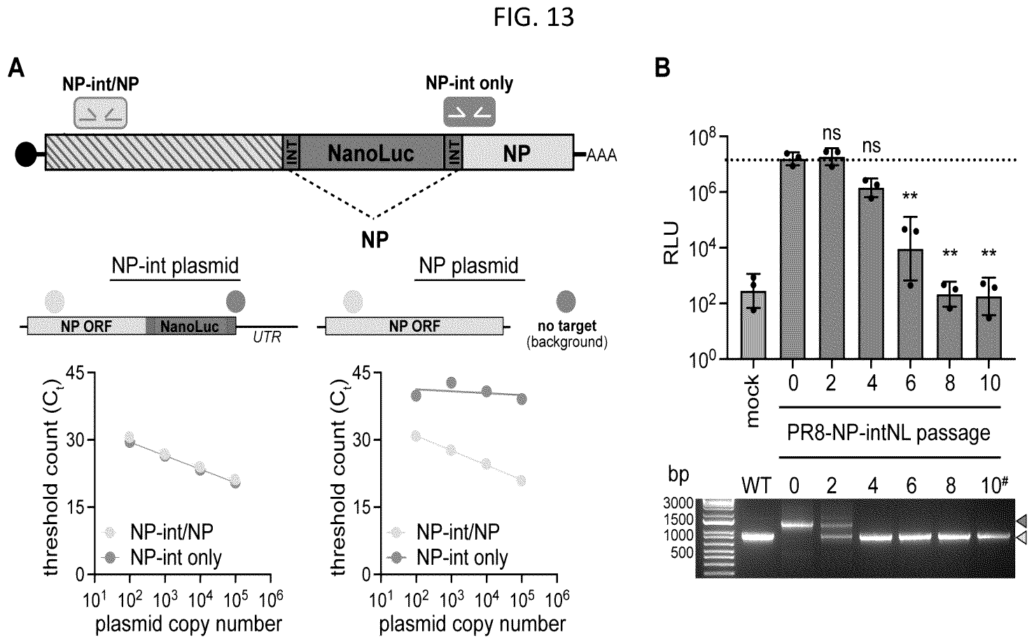

We were next interested to see if an artificial NanoLuc encoding intron inserted into a different viral genomic locus would be viable and if the resulting virus would have similar characteristics to the NA-intNL virus. We therefore incorporated the intron-sequence-flanked NanoLuc reporter into segment 5, which encodes the NP protein, using the same insertion scheme as for segment 6 (FIG. 6A). Indeed, we were able to rescue a PR8-NP-intNL virus which grew to high titers, though with delayed kinetics and ultimately reduced titers relative to WT virus, on MDCK cells (FIG. 6B-E). In contrast to the NA-intNL mRNA, the NP-intNL mRNA only spliced ˜80% of the time (FIG. 6F and FIG. 13A). High luciferase signal was detected after infection, and the luciferase levels correlated well with viral MOI (FIG. 6G). To define the stability of the reporter at this viral locus, we performed serial passaging on MDCK cells. In contrast to the NA-intNL virus, we observed a loss in reporter signal, segment length and NanoLuc sequence after several rounds of serial passage (FIG. 13B). Thus, not all intronic insertion sites produce viruses with the same characteristics, and some of these genomic modifications impart a significant defect on viral fitness.

One potential benefit of utilizing a non-glycoprotein encoding intron insertion site such as segment 5 is that segments 4 and 6 can be exchanged with corresponding segments from other strains. These so-called “6+2” reassortants (harboring internal segments from a laboratory adapted strain such as PR8 and the glycoprotein segments from a contemporary strain) are frequently generated to improve vaccine yields or to facilitate growth in animal models of infection [39]. To show that PR8 NP segments harboring reporter introns have utility for this approach, we generated a virus with the glycoproteins from the recently characterized H1N1 G4 swine virus A/swine/Henan/SN13/2018 (SW/HN/SN13/18, SW18) [40] along with the 6 remaining segments from PR8 (FIG. 611). The G4/PR8-NP-intNL virus grew to high titers and produced similar luciferase activity levels compared to PR8-NP-intNL virus (FIG. 6I-K), demonstrating the potential utility of this approach.

### Intron-Based Reporters are a Generalizable Approach for the Development of Reporter Influenza Virus Strains

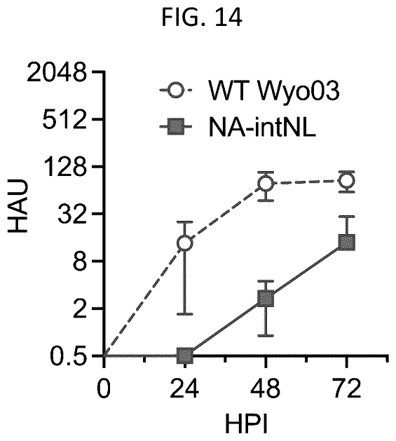

Finally, we were interested in testing if our newfound knowledge regarding IAV tolerance of artificial introns could be leveraged as a generalizable platform to generate reporter influenza viruses. We therefore selected an H3N2 IAV, A/Wyoming/03/2003 (Wyo/03) that is highly divergent from PR8. We then developed a set of design guidelines based on all of the data we had previously generated (FIG. 7A). First, we recommend selecting a normally nonsplicing segment as well as one without multiple overlapping reading frames to theoretically maximize productive reporter translation. We selected the Wyo/03 NA segment because while that protein is of a different subtype and unrelated to the the PR8 NA, the NP segment is reasonably conserved between the two viruses. In our case, we identified the nucleotide sequence “AAGGUG” in the Wyo/03 NA ORF; however, if a viral segment does not contain the nucleotide sequence “AAGGUG,” it may be introduced using silent mutations (Table 5). We then inserted the intron-flanked NanoLuc sequence between the “AAG” and “GUG” nucleotides and verified that NanoLuc was in the correct reading frame. Finally, we rescued the Wyo/03-NA-intNL virus on MDCK cells (FIG. 7B). The Wyo/03-NA-intNL virus grew to lower titers compared to WT Wyo/03 H3N2 virus (FIG. 7C and FIG. 14). However, we detected significant luciferase activity during Wyo/03-NA-intNL virus infection of MDCK cells demonstrating the successful translation of the unspliced reporter-encoding Wyo/03 NA-intNL mRNA (FIG. 7D). Thus, including an intron reporter sequence in a normally intronless IAV segment is a viable, and likely broadly generalizable, method for producing novel reporter viruses.

TABLE 5
Inserting splicing environments
into non-splicing influenza virus
segments using silent mutations

| | Amino | |
|---|---|---|
| | Acid | |
| | Sequence | New Nucleotide Sequence |
| 1 | KV | AAG GTG |
| 2 | ARC | GCA AGG TG[T,C] |
| 3 | ARW | GCA AGG TGG |
| 4 | ERC | GAA AGG TGIT,C] |
| 5 | ERW | GAA AGG TGG |
| 6 | GRC | GGA AGG TG[T, C] |
| 7 | GRW | GGA AGG TGG |
| 8 | IRC | ATA AGG TG[T,C] |
| 9 | IRW | ATA AGG TGG |
| 10 | KRC | AAA AGG TG[T,C] |
| 11 | KRW | AAA AGG TGG |
| 12 | LRC | TTA AGG TG[T, C] |
| 13 | LRW | TTA AGG TGG |
| 14 | PRC | CCA AGG TG[T,C] |
| 15 | PRW | CCA AGG TGG |
| 16 | QRC | CAA AGG TG[T,C] |
| 17 | QRW | CAA AGG TGG |
| 18 | RRC | CGA AGG TG[T,C] |
| 19 | RRW | CGA AGG TGG |
| 20 | SRC | TCA AGG TG[T,C] |
| 21 | SRW | TCA AGG TGG |
| 22 | TRC | ACA AGG TG[T, C] |
| 23 | TRW | ACA AGG TGG |
| 24 | VRC | GTA AGG TG[T, C] |
| 25 | VRW | GTA AGG TGG |
| 26 | EGA | GAA GGT GC[T, C, A, G] |
| 27 | EGD | GAA GGT GA[T, C] |
| 28 | EGE | GAA GGT GA[A, G] |
| 29 | EGG | GAA GGT GG[T, C, A, G] |
| 30 | EGV | GAA GGT GT[T, C, A, G] |
| 31 | KGA | AAA GGT GC[T, C, A, G] |
| 32 | KGD | AAA GGT GA[T, C] |
| 33 | KGE | AAA GGT GA[A, G] |
| 34 | KGG | AAA GGT GG[T, C, A, G] |
| 35 | KGV | AAA GGT GT[T, C, A, G] |
| 36 | QGA | CAA GGT GC[T, C, A, G] |
| 37 | QGD | CAA GGT GA[T, C] |
| 38 | QGE | CAA GGT GA[A, G] |
| 39 | QGG | CAA GGT GG[T, C, A, G] |
| 40 | QGV | CAA GGT GT[T, C, A, G] |

### Discussion

Influenza viruses take advantage of host splicing machinery to produce multiple functional proteins from a single viral segment. In this Example, we explored the constraints on IAV genomic splicing and leveraged our findings to generate IAV reporter strains by introducing intronic reporters into otherwise nonsplicing viral segments. Overall, this work demonstrates that adapting a viral method of host hijacking, specifically taking advantage of the host splicing machinery, and applying it to additional segments is both a permissible and practical method for expanding the coding capacity of influenza viruses.

We demonstrated how artificial introns can be used for the generation of novel influenza reporter viruses. One advantage of this approach over previous luciferase reporter influenza viruses is that RNA packaging signal mapping and/or manipulation is not required [48]. Furthermore, it may be possible to introduce introns into multiple segments and produce multiple reporter proteins at the same time. Another benefit of the system is its flexibility. We have already shown that PR8 segments NA and NP tolerate the intronic reporters. Using the Influenza Research Database, we found among searchable IAV NA and NP sequences, greater than 100,000 segments contained amino acid sequences compatible with introducing the “AAGGUG” nucleotide sequences [49]. This analysis demonstrates our design guidelines for incorporating intronic reporters to produce novel reporter influenza viruses are widely applicable.

However, there are additional considerations when generating a reporter virus by inserting an intron. First, as currently constructed, the reporter is fused to the 5′ sequence of the viral gene ORF. For example, for our NA-intNL viruses, NanoLuc is fused with the stalk domain and is potentially trafficked to the membrane and incorporated into the virion, likely reducing viral fitness. To prevent this fusion, a 2A cleavage-site could be incorporated ahead of the reporter reading frame. Another aspect of consideration is codon usage as introns are partially identified by their different GC content compared to their adjacent exons [50]. Since IAVs have low GC content relative to their hosts [51,52], it is potentially important to consider how the primary sequence of the intron relates to the viral background. Overall, with these considerations in mind, we believe inserting intronic reporters into intronless viral segments is a promising, generalizable way to generate new influenza reporter viruses.

In sum, we sought to learn whether additional IAV segments could tolerate splicing. By experimental introduction of artificial introns, we found that not only were introns tolerated, but they could be used to express additional proteins. While we leveraged these observations to generate viral reporter strains, the approaches described in this work represent new tools that may be able to aid in understanding the mechanisms that normally underly splicing in the influenza genome. Future rational use of artificial introns to modify influenza viral genomes has broad utility and will facilitate investigation into molecular virology, viral pathogenesis, and translational research questions.

### REFERENCES

1. Firth A E, Brierley I. Non-canonical translation in RNA viruses. J Gen Virol [Internet]. 2012. [cited 2020 Nov. 23]; 93 (PART 7):1385-409.
2. Whelan S. Viral replication strategies. In: Fields Virology: Sixth Edition. Wolters Kluwer Health Adis (ESP); 2013.
3. Dou D, Revol R, Östbye H, Wang H, Daniels R. Influenza A Virus Cell Entry, Replication, Virion Assembly and Movement. Front Immunol [Internet]. 2018. Jul. 20 [cited 2020 Nov. 4]; 9:1581.
4. Dubois J, Terrier 0, Rosa-Calatrava M. Influenza viruses and mRNA splicing: doing more with less. MBio [Internet]. 2014. May 13 [cited 2020 Sep. 5]; 5(3):e00070-14.
5. Robb N C, Jackson D, Vreede F T, Fodor E. Splicing of influenza A virus NS1 mRNA is independent of the viral NS1 protein. J Gen Virol [Internet]. 2010. [cited 2020 Nov. 13]; 91(9):2331-40.
6. Krug R M. Functions of the influenza A virus NS1 protein in antiviral defense. Curr Opin Virol [Internet]. 2015. Jun. 1 [cited 2020 Nov. 30]; 12:1-6.
7. Valcarcel J, Portela A, Ortin J. Regulated M1 mRNA splicing in influenza virus-infected cells [Internet]. Vol. 72, Journal of General Virology. 1991. [cited 2020 Nov. 13].
8. Manzoor R, Igarashi M, Takada A. Influenza A Virus M2 Protein: Roles from Ingress to Egress. Int J Mol Sci [Internet]. 2017. Dec. 7 [cited 2020 Nov. 30]; 18(12).
9. Ren X, Yu Y, Li H, Huang J, Zhou A, Liu S, et al., Avian Influenza A Virus Polymerase Recruits Cellular RNA Helicase eIF4A3 to Promote Viral mRNA Splicing and Spliced mRNA Nuclear Export. Front Microbiol [Internet]. 2019. Jul. 16 [cited 2020 Aug. 4]; 10:1625.
10. Huang X, Zheng M, Wang P, Mok B W-Y, Liu S, Lau S-Y, et al., An NS-segment exonic splicing enhancer regulates influenza A virus replication in mammalian cells. Nat Commun [Internet]. 2017. [cited 2020 Aug. 19]; 8:14751.
11. Zhu Y, Wang R, Yu L, Sun H, Tian S, Li P, et al., Human TRA2A determines influenza A virus host adaptation by regulating viral mRNA splicing. Sci Adv [Internet]. 2020. Jun. 19 [cited 2020 Aug. 4]; 6(25):eaaz5764.
12. Calderon B M, Danzy S, Delima G K, Jacobs N T, Ganti K, Hockman M R, et al., Dysregulation of M segment gene expression contributes to influenza A virus host restriction. Schultz-Cherry S, editor. PLOS Pathog [Internet]. 2019. Aug. 15 [cited 2020 Aug. 4]; 15(8):e1007892.
13. Bogdanow B, Wang X, Eichelbaum K, Sadewasser A, Husic I, Paki K, et al., The dynamic proteome of influenza A virus infection identifies M segment splicing as a host range determinant. Nat Commun [Internet]. 2019. Dec. 4 [cited 2020 Aug. 4]; 10(1):5518.
14. Jackson D, Lamb R A. The influenza A virus spliced messenger RNA M mRNA3 is not required for viral replication in tissue culture. [cited 2020 Nov. 23];
15. Selman M, Dankar S K, Forbes N E, Jia J-J, Brown E G. Adaptive mutation in influenza A virus non-structural gene is linked to host switching and induces a novel protein by alternative splicing. Emerg Microbes Infect [Internet]. 2012. Jul. 25 [cited 2020 Aug. 4]; 1(1):1-10.
16. Shih S-R, Suen P-C, Chen Y-S, Chang S-C. A Novel Spliced Transcript of Influenza A/WSN/33 Virus. Virus Genes [Internet]. 1998. [cited 2020 Nov. 23]; 17(2):179-83.
17. Reuther P, Göpfert K, Dudek A H, Heiner M, Herold S, Schwemmle M. Generation of a variety of stable Influenza A reporter viruses by genetic engineering of the NS gene segment. Sci Rep [Internet]. 2015. Sep. 12 [cited 2020 Nov. 23]; 5(1):11346.
18. Manicassamy B, Manicassamy S, Belicha-Villanueva A, Pisanelli G, Pulendran B, Garcia-Sastre A. Analysis of in vivo dynamics of influenza virus infection in mice using a GFP reporter virus. Proc Natl Acad Sci USA [Internet]. 2010. [cited 2020 Nov. 23]; 107(25):11531-6.
19. Nogales A, DeDiego M L, Topham D J, Martinez-Sobrido L. Rearrangement of Influenza Virus Spliced Segments for the Development of Live-Attenuated Vaccines. J Virol [Internet]. 2016. Jul. 15 [cited 2020 Nov. 17]; 90(14):6291-302.
20. Shaw M L, Palese P. Orthomyxoviridae. In: Fields Virology: Sixth Edition. Wolters Kluwer Health Adis (ESP); 2013.
21. Yamayoshi S, Watanabe M, Goto H, Kawaoka Y. Identification of a Novel Viral Protein Expressed from the PB2 Segment of Influenza A Virus. J Virol [Internet]. 2016. Jan. 1 [cited 2020 Aug. 16]; 90(1):444-56.
22. Bonano V I, Oltean S, Garcia-Blanco M A. A protocol for imaging alternative splicing regulation in vivo using fluorescence reporters in transgenic mice. Nat Protoc [Internet]. 2007. Sep. 30 [cited 2020 Nov. 9]; 2(9):2166-81.
23. Valcarcel J, Portela A, Ortin J. Regulated M1 mRNA splicing in influenza virus-infected cells. J Gen Virol [Internet]. 1991. [cited 2020 Oct. 23]; 72(6):1301-8.
24. Lau S C, Scholtissek C. Abortive Infection of Vero Cells by an Influenza A Virus (FPV). Virology [Internet]. 1995. Sep. 10 [cited 2020 Nov. 23]; 212(1):225-31.
25. Inglis S C, Brown C M. Differences in the Control of Virus mRNA Splicing during Permissive or Abortive Infection with Influenza A (Fowl Plague) Virus [Internet]. Vol. 65, J. gen. Virol. 1984. [cited 2020 Nov. 23].
26. Dvinge H. Regulation of alternative mRNA splicing: old players and new perspectives. FEBS Lett [Internet]. 2018. Sep. 16 [cited 2020 Nov. 24]; 592(17):2987-3006.
27. Fox-Walsh K L, Dou Y, Lam B J, Hung S-P, Baldi P F, Hertel K J. The architecture of pre-mRNAs affects mechanisms of splice-site pairing [Internet]. Vol. 8. 2005. [cited 2020 Nov. 9].
28. England C G, Ehlerding E B, Cai W. NanoLuc: A Small Luciferase Is Brightening Up the Field of Bioluminescence. Bioconjug Chem [Internet]. 2016. [cited 2020 Nov. 9]; 27(5):1175-87.
29. Hall M P, Unch J, Binkowski B F, Valley M P, Butler B L, Wood M G, et al., Engineered Luciferase Reporter from a Deep Sea Shrimp Utilizing a Novel Imidazopyrazinone Substrate. ACS Chem Biol [Internet]. 2012. Nov. 16 [cited 2020 Nov. 9]; 7(11):1848-57.
30. Heaton N S, Leyva-Grado V H, Tan G S, Eggink D, Hai R, Palese P. In vivo bioluminescent imaging of influenza a virus infection and characterization of novel cross-protective monoclonal antibodies. J Virol [Internet]. 2013. Aug. 1 [cited 2020 Sep. 5]; 87(15):8272-81.
31. Yan D, Weisshaar M, Lamb K, Chung H K, Lin M Z, Plemper R K. Replication-Competent Influenza Virus and Respiratory Syncytial Virus Luciferase Reporter Strains Engineered for Co-Infections Identify Antiviral Compounds in Combination Screens. Biochemistry [Internet]. 2015. Sep. 15 [cited 2020 Nov. 24]; 54(36):5589-604. 10.1021/acs.biochem.5b00623
32. Karlsson E A, Meliopoulos V A, Savage C, Livingston B, Mehle A, Schultz-Cherry S. Visualizing real-time influenza virus infection, transmission and protection in ferrets. Nat Commun [Internet]. 2015. Mar. 6 [cited 2020 Nov. 24]; 6:6378.
33. Pan W, Dong Z, Li F, Meng W, Feng L, Niu X, et al., Visualizing influenza virus infection in living mice. Nat Commun [Internet]. 2013. [cited 2020 Nov. 13]; 4:2369.
34. Sutton T C, Obadan A, Lavigne J, Chen H, Li W, Perez D R. Genome rearrangement of influenza virus for anti-viral drug screening. Virus Res [Internet]. 2014. Aug. 30 [cited 2020 Nov. 24]; 189:14-23.
35. Eckert N, Wrensch F, Gartner S, Palanisamy N, Goedecke U, Jäger N, et al., Influenza A Virus Encoding Secreted Gaussia Luciferase as Useful Tool to Analyze Viral Replication and Its Inhibition by Antiviral Compounds and Cellular Proteins. PLoS One [Internet]. 2014. [cited 2020 Nov. 24]; 9(5).
36. Noshi T, Kitano M, Taniguchi K, Yamamoto A, Omoto S, Baba K, et al., In vitro characterization of baloxavir acid, a first-in-class cap-dependent endonuclease inhibitor of the influenza virus polymerase PA subunit. Antiviral Res [Internet]. 2018. Dec. 1 [cited 2020 Aug. 30]; 160:109-17.
37. Reale M A, Manheimer A J, Moran T_{M}, Norton G, Bona C A, Schulman J L. Characterization of monoclonal antibodies specific for sequential influenza A/PR/8/34 virus variants. J Immunol [Internet]. 1986. Aug. 15 [cited 2021 Apr. 12]; 137(4):1352-8.
38. Urbanski L M, Leclair N, Anczuków O. Alternative-splicing defects in cancer: Splicing regulators and their downstream targets, guiding the way to novel cancer therapeutics. Wiley Interdiscip Rev RNA [Internet]. 2018. [cited 2020 Nov. 18]; 9(4):e1476.
39. Nogales A, Martinez-Sobrido L. Reverse genetics approaches for the development of influenza vaccines. Int J Mol Sci. 2017; 18(1):1-26.
40. Sun H, Xiao Y, Liu J, Wang D, Li F, Wang C, et al., Prevalent Eurasian avian-like H1N1 swine influenza virus with 2009 pandemic viral genes facilitating human infection. Proc Natl Acad Sci USA [Internet]. 2020 Jul. 21 [cited 2021 Apr. 12]; 117(29):17204-10.
41. Shih S R, Krug R M. Novel exploitation of a nuclear function by influenza virus: the cellular SF2/ASF splicing factor controls the amount of the essential viral M2 ion channel protein in infected cells. EMBO J [Internet]. 1996. Oct. 1 [cited 2020 Nov. 24]; 15(19):5415-27.
42. Gultyaev A P, Heus H A, Olsthoorn R C L. An RNA conformational shift in recent H5N1 influenza A viruses. Bioinformatics [Internet]. 2007. Feb. 1 [cited 2020 Nov. 24]; 23(3):272-6.
43. Moss W N, Dela-Moss L I, Priore S F, Turner D. The influenza A segment 7 mRNA 3′ splice site pseudoknot/hairpin family. RNA Biol [Internet]. 2012. Nov. 27 [cited 2020 Nov. 24]; 9(11):1305-10.
44. Moss W N, Dela-Moss L I, Kierzek E, Kierzek R, Priore S F, Turner D H. The 3′ Splice Site of Influenza A Segment 7 mRNA Can Exist in Two Conformations: A Pseudoknot and a Hairpin. Lewin A, editor. PLoS One [Internet]. 2012. Jun. 7 [cited 2020 Nov. 24]; 7(6):e38323.
45. Jiang T, Nogales A, Baker S F, Martinez-Sobrido L, Turner D H. Mutations Designed by Ensemble Defect to Misfold Conserved RNA Structures of Influenza A Segments 7 and 8 Affect Splicing and Attenuate Viral Replication in Cell Culture. Pohlmann S, editor. PLoS One [Internet]. 2016. Jun. 7 [cited 2020 Sep. 9]; 11(6):e0156906.
46. Garaigorta U, Ortin J. Mutation analysis of a recombinant NS replicon shows that influenza virus NS1 protein blocks the splicing and nucleo-cytoplasmic transport of its own viral mRNA. Nucleic Acids Res [Internet]. 2007. Jun. 25 [cited 2020 Nov. 24]; 35(14):4573-82.
47. Shih S-R, Nemeroff M E, Krug R M. The choice of alternative 5′ splice sites in influenza virus Ml mRNA is regulated by the viral polymerase complex [Internet]. Vol. 92, Biochemistry. 1995. [cited 2020 Nov. 24].
48. Breen M, Nogales A, Baker S F, Martinez-Sobrido L. Replication-Competent Influenza A Viruses Expressing Reporter Genes. Viruses [Internet]. 2016. [cited 2020 Nov. 15]; 8(7).
49. Zhang Y, Aevermann B D, Anderson T K, Burke D F, Dauphin G, Gu Z, et al., Influenza Research Database: An integrated bioinformatics resource for influenza virus research. Nucleic Acids Res [Internet]. 2017. [cited 2020 Nov. 24]; 45(D1):D466-74.
50. Amit M, Donyo M, Hollander D, Goren A, Kim E, Gelfman S, et al., Differential G C Content between Exons and Introns Establishes Distinct Strategies of Splice-Site Recognition. Cell Rep [Internet]. 2012. May 31 [cited 2020 Aug. 4]; 1(5):543-56.
51. Auewarakul P. Composition bias and genome polarity of RNA viruses. Virus Res [Internet]. 2005. Apr. [cited 2020 Dec. 1]; 109(1):33-7.
52. Fancher K C, Hu W. Codon bias of influenza a viruses and their hosts. Am J Mol Biol [Internet]. 2011. [cited 2020 Dec. 1]; 1:174-82.

### Example 2

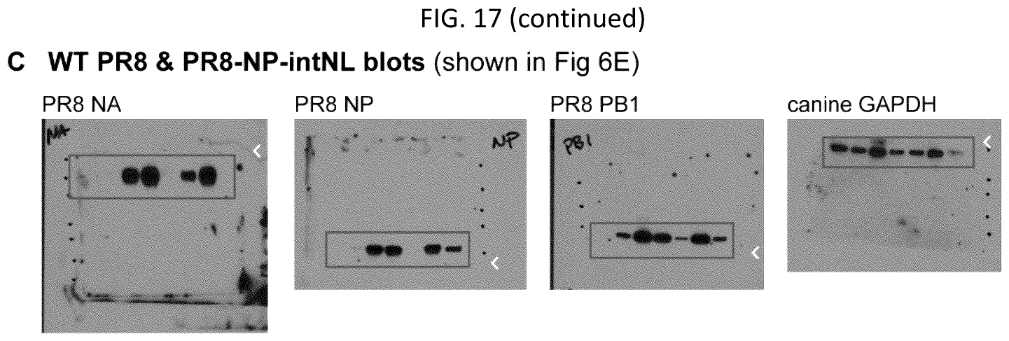

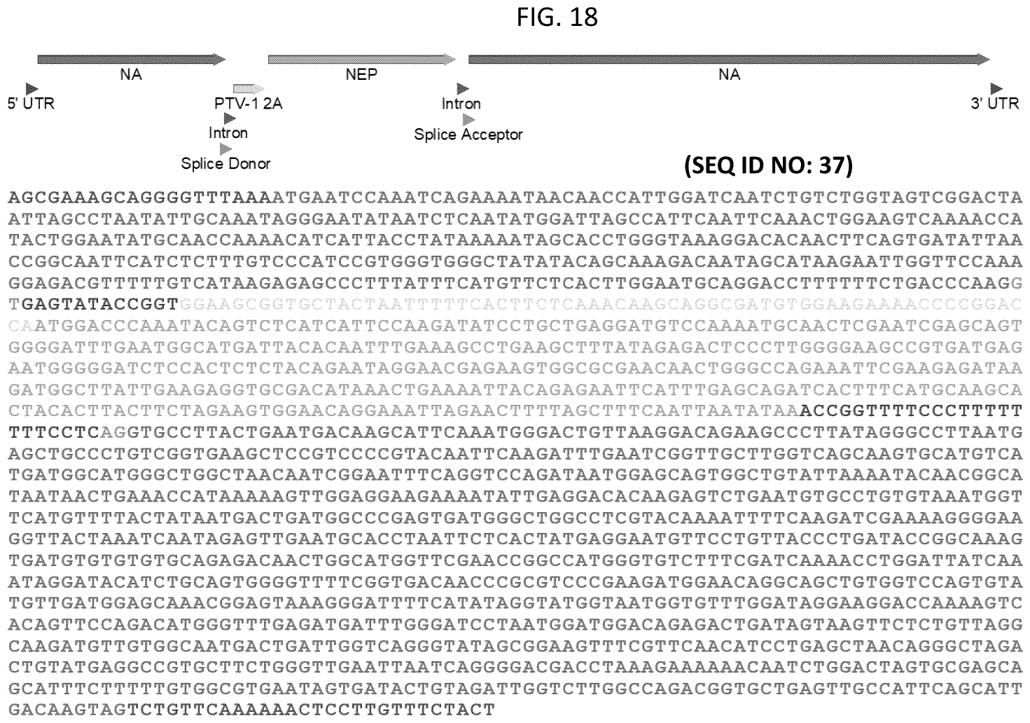

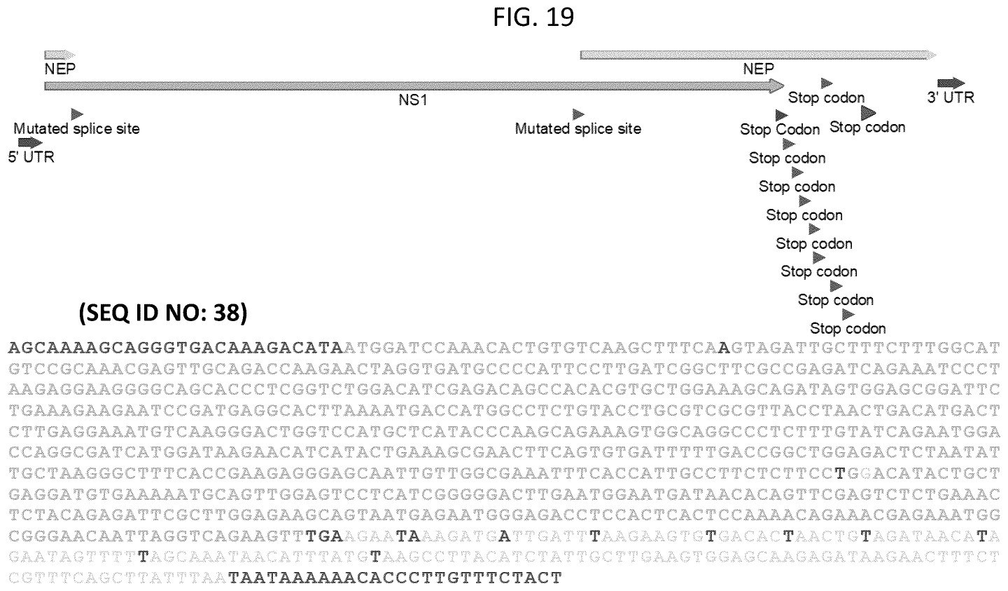

In the following example, the inventors describe the generation of a A/Puerto Rico/8/1934 (PR8) influenza A virus comprising an artificial intron in segment 6 that encodes the influenza protein nuclear export protein (NEP) (also referred to as NS2; see FIG. 18), which is natively expressed from viral segment 8. This virus also comprises mutations in segment 8 that disrupt the splicing and expression of the native NEP gene (FIG. 19).

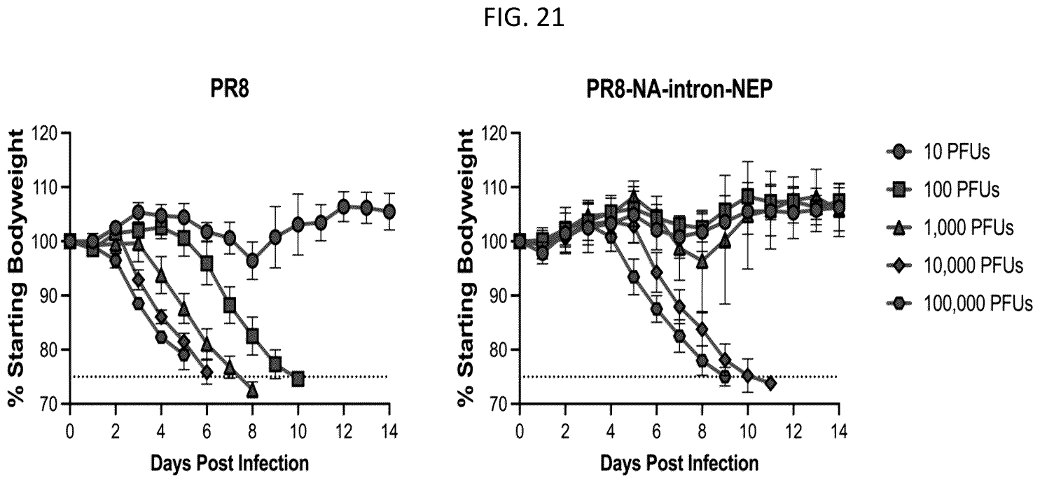

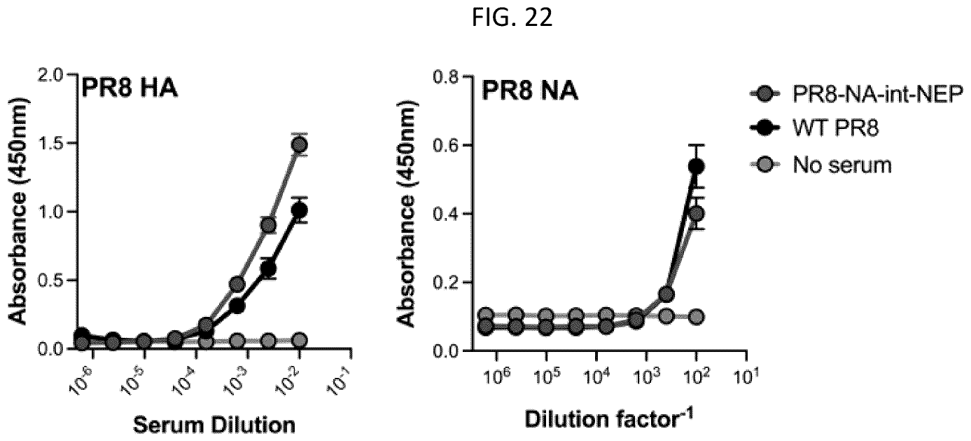

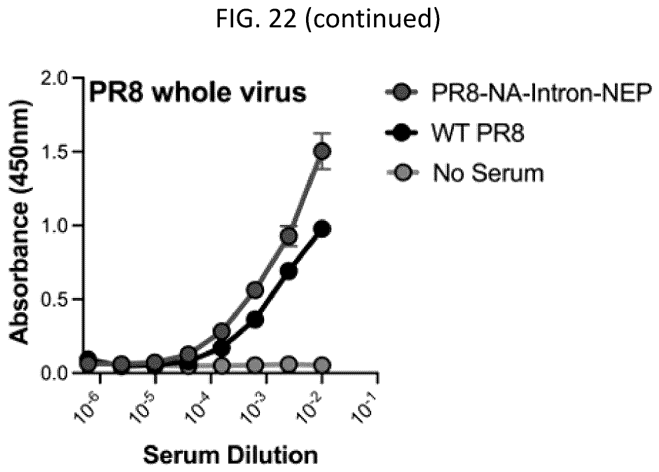

To understand the attenuation of the PR8-NA-intron-NEP live attenuated vaccine virus in a mouse model, 8-week old C57BL/6J mice were infected intranasally with either wild-type (WT) PR8 or PR8-NA-intron-NEP virus at varying doses. Bodyweights were monitored and recorded for 14 days post infection. The results show that mice infected with >10 PFU of WT PR8 virus succumb to virus (FIG. 21). However, infection with PR8-NA-intron-NEP is tolerated up to a dosage of 1,000 PFU. This indicates a 100-fold attenuation in virulence of the PR8-NA-intron-NEP virus compared to WT virus. Additionally, ELISAs against soluble PR8 HA protein, soluble PR8 NA protein, and whole virus using sera from the highest surviving dosage group of each virus (10 PFU WT PR8 or 1,000 PFU PR8-NA-intron-NEP) reveal that infection with PR8-NA-intron-NEP elicits a greater immune response against PR8 HA and PR8 whole virus than infection with WT PR8 (FIG. 22). With regards to the immune response against the NA glycoprotein, PR8-NA-intron-NEP elicited a lower immune response against PR8 NA than WT PR8, which is attributed to decreased expression of NA arising from the manipulation of this genomic segment (FIG. 22). These results demonstrate that infection with PR8-NA-intron-NEP induces a more robust immune response than infection with WT PR8, and that this immune response is biased towards the HA glycoprotein.

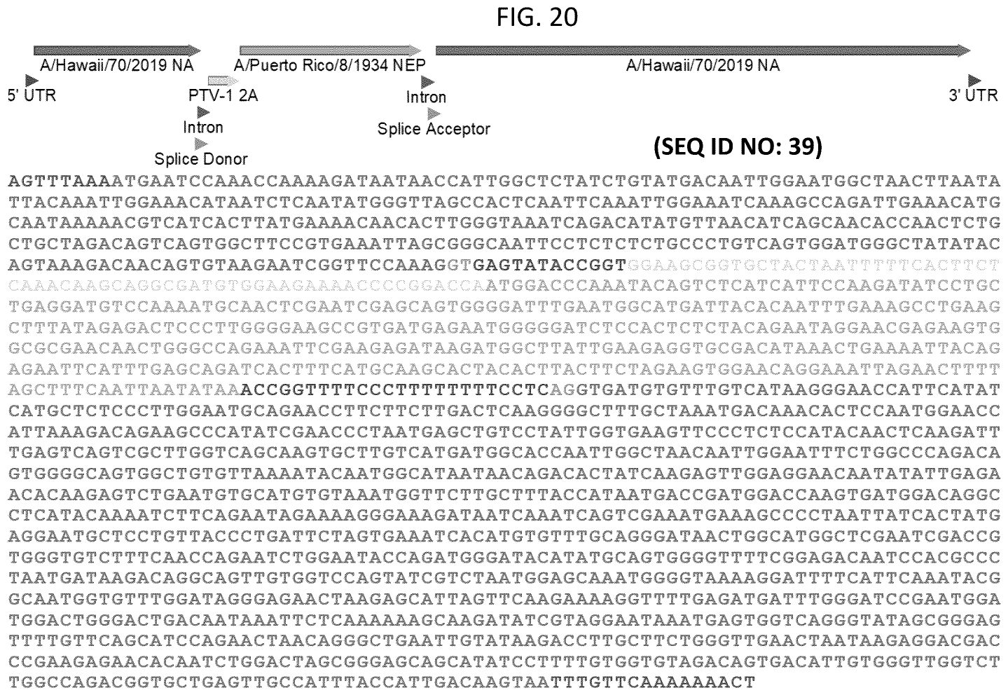

To demonstrate that artificial introns can also be used to introduce antigens from other viruses, the same artificial intron comprising the PR8 NEP protein was also introduced into segment 6 of the influenza A strain A/Hawaii/70/2019 (FIG. 20). This virus represents a 6+2 reassortant that could be used for commercial vaccine production. This same kind of approach would be used to generate live attenuated viruses, putting clinically relevant glycoproteins into a “platform” genetic background and encoding the matched platform NEP in the relevant segment 6.

1. A recombinant viral segment comprising a viral segment from a negative-strand RNA virus into which an artificial intron has been inserted, wherein the 3′ end of the artificial intron comprises the sequence AC and forms a 3′ splice site with the upstream portion of the viral segment, and the 5′ end of the artificial intron comprises the sequence CU and forms a 5′ splice site with the downstream portion of the viral segment, wherein the artificial intron comprises a branch site 20-50 bases downstream of the 5′ end, and wherein the viral segment is from a virus in the Orthomyxoviridae or Bornaviridae family.

2. The recombinant viral segment of claim 1, wherein:
a) the 3′ end of the artificial intron comprises the sequence ACUYAC;
b) the 5′ end of the artificial intron comprises the sequence CUGN followed by a 13-20 nucleotide long purine-rich region; and/or
c) the branch site comprises the sequence RUYAR.

3. The recombinant viral segment of claim 2, wherein:
a) the 3′ end of the artificial intron comprises the sequence ACUCAC;
b) the 5′ end of the artificial intron comprises the sequence CUGAGGAAAAAAAAGGGAAAA (SEQ ID NO: 25); and/or
c) the branch site comprises the sequence GUUAG.

4. (canceled)

5. (canceled)

6. (canceled)

7. (canceled)

8. The recombinant viral segment of claim 1, wherein the artificial intron is inserted into the viral segment such that it is flanked on the 3′ end by the sequence CUK and is flanked on the 5′ end by the sequence NNC.

9. (canceled)

10. The recombinant viral segment of claim 8, wherein the artificial intron is inserted into the genome segment such that it is flanked on the 3′ end by the sequence CUU and is flanked on the 5′ end by the sequence CAC.

11. The recombinant viral segment of claim 1, wherein the artificial intron encodes a protein of interest, wherein the artificial intron is inserted into an open reading frame (ORF) of a viral gene within the viral segment, and wherein the artificial intron is inserted such that the portion of the artificial intron encoding the protein of interest is in the same reading frame as the ORF.

12. The recombinant viral segment of claim 11, wherein the protein of interest is a reporter protein, an antigen from a different segment of the virus, or an antigen from a different virus.

13. (canceled)

14. (canceled)

15. The recombinant viral segment of claim 11, wherein the artificial intron further encodes a linker on the N-terminal end of the protein of interest.

16. The recombinant viral segment of claim 1, wherein the viral segment is not natively spliced and does not contain multiple overlapping reading frames.

17. The recombinant viral segment of claim 1, wherein the negative-strand RNA virus is an influenza A virus.

18. The recombinant viral segment of claim 17, wherein the viral segment is selected from segment 5 or 6.

19. (canceled)

20. The recombinant viral segment of claim 1, wherein the artificial intron comprises SEQ ID NO: 28, 31, or 34.

21. A DNA construct comprising a polynucleotide encoding the recombinant viral segment of claim 1.

22. (canceled)

23. A virus comprising the recombinant viral segment of claim 1, wherein the virus is a negative-strand RNA virus from the Orthomyxoviridae or Bornaviridae family.

24. (canceled)

25. A method of making a virus comprising an artificial intron, the method comprising rescuing the virus with the DNA construct of claim 21.

26. A method of using virus of claim 23 in a screening assay.

27. (canceled)

28. A method of inducing an immune response in a subject, the method comprising administering the virus of claim 23 to the subject.

29. The method of claim 28, wherein:
a) the immune response is against the virus,
b) the artificial intron encodes a protein of interest, and the immune response is against the protein of interest: or
c) both a) and b).

30. (canceled)

31. (canceled)

32. A method of delivering a protein of interest to a cell in a subject, the method comprising administering the virus of claim 23 to the subject, wherein the virus comprises an artificial intron that encodes the protein of interest.

33. A method of generating a recombinant negative-strand RNA virus from the Orthomyxoviridae or Bornaviridae family comprising:
a) selecting a nonsplicing, non-frameshifting segment of the virus;
b) identifying or introducing a nucleotide sequence that can act as a splice site into the viral segment;
c) introducing an artificial intron into a construct encoding the viral segment at the splice site, wherein the artificial intron contains a nucleotide sequence encoding a protein or RNA of interest in frame with the virally encoded protein flanked by a 5′ intron end and a 3′ intron end and a branch site;
d) introducing the construct of step (c) into a viral rescue plasmid; and
e) rescuing the virus by transfecting cells with the viral rescue plasmid of step (d).
